(19) United States
(12) Patent Application Publication
(10) Pub. No.: US 2015/0313530 A1
(43) Pub. Date: Nov. 5, 2015

(54) MENTAL STATE EVENT DEFINITION GENERATION

(71) Applicant: Affectiva, Inc., Waltham, MA (US)

(72) Inventors: Evan Kodra, Waltham, MA (US); Rana el Kaliouby, Milton, MA (US); Thomas James Vandal, Dracut, MA (US)

(21) Appl. No.: 14/796,419

(22) Filed: Jul. 10, 2015

Related U.S. Application Data
(63) Continuation-in-part of application No. 13/153,745, filed on Jun. 6, 2011.
Application No. 13/153,745 is a continuation-in-part of application No. 14/460,915, filed on Aug. 15, 2014.
Application No. 14/460,915 is a continuation-in-part of application No. 13/153,745, filed on Jun. 6, 2011.
(60) Provisional application No. 62/023,800, filed on Jul. 11, 2014.
(60) Provisional application No. 62/047,508, filed on Sep. 8, 2014.
(60) Provisional application No. 62/082,579, filed on Nov. 20, 2014.
(60) Provisional application No. 62/128,974, filed on Mar. 5, 2015.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/867,007, filed on Aug. 16, 2013.
(60) Provisional application No. 61/924,252, filed on Jan. 7, 2014.
(60) Provisional application No. 61/916,190, filed on Dec. 14, 2013.
(60) Provisional application No. 61/927,481, filed on Jan. 15, 2014.
(60) Provisional application No. 61/953,878, filed on Mar. 16, 2014.
(60) Provisional application No. 61/972,314, filed on Mar. 30, 2014.
(60) Provisional application No. 62/023,800, filed on Jul. 11, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. A61B 5/16; G06K 9/46; G06K 9/00; G06K 9/62; A61B 5/00; G09B 5/06

(52) U.S. Cl. CPC A61B 5/165; A61B 5/0077; A61B 5/7264; G09B 5/06; G06K 9/00302; G06K 9/6218; G06K 9/00718; G06K 9/4604; G06K 9/4642; G06K 2009/485

(57) ABSTRACT

Analysis of mental states is provided based on videos of a plurality of people experiencing various situations such as media presentations. Videos of the plurality of people are captured and analyzed using classifiers. Facial expressions of the people in the captured video are clustered based on set criteria. A unique signature for the situation to which the people are being exposed is then determined based on the expression clustering. In certain scenarios, the clustering is augmented by self-report data from the people. In embodiments, the expression clustering is based on a combination of multiple facial expressions.

### RELATED APPLICATIONS

This application claims the benefit of U.S. provisional patent applications “Mental State Event Definition Generation” Ser. No. 62/023,800, filed Jul. 11, 2014, “Facial Tracking with Classifiers” Ser. No. 62/047,508, filed Sep. 8, 2014, “Semiconductor Based Mental State Analysis” Ser. No. 62/082,579, filed Nov. 20, 2014, and “Viewership Analysis Based On Facial Evaluation” Ser. No. 62/128,974, filed Mar. 5, 2015. This application is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. This application is also a continuation-in-part of U.S. patent application “Mental State Analysis Using an Application Programming Interface” Ser. No. 14/460,915, Aug. 15, 2014, which claims the benefit of U.S. provisional patent applications “Application Programming Interface for Mental State Analysis” Ser. No. 61/867,007, filed Aug. 16, 2013, “Mental State Analysis Using an Application Programming Interface” Ser. No. 61/924,252, filed Jan. 7, 2014, “Heart Rate Variability Evaluation for Mental State Analysis” Ser. No. 61/916,190, filed Dec. 14, 2013, “Mental State Analysis for Norm Generation” Ser. No. 61/927,481, filed Jan. 15, 2014, “Expression Analysis in Response to Mental State Express Request” Ser. No. 61/953,878, filed Mar. 16, 2014, “Background Analysis of Mental State Expressions” Ser. No. 61/972,314, filed Mar. 30, 2014, and “Mental State Event Definition Generation” Ser. No. 62/023,800, filed Jul. 11, 2014; the application is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. The foregoing applications are each hereby incorporated by reference in their entirety.

### FIELD OF ART

This application relates generally to mental state analysis and more particularly to mental state event definition generation.

### BACKGROUND

Individuals have mental states that vary in response to various situations in life. While an individual's mental state is important to general well-being and impacts his or her decision making, multiple individuals' mental states resulting from a common event can carry a collective importance that, in certain situations, is even more important than an individual's mental state. Mental states include a wide range of emotions and experiences from happiness to sadness, from contentedness to worry, from excitation to calm, and many others. Despite the importance of mental states in daily life, the mental state of even a single individual might not always be apparent, even to the individual. In fact, the ability and means by which one person perceives his or her emotional state can be quite difficult to summarize. Though an individual can often perceive his or her own emotional state quickly, instinctively and with a minimum of conscious effort, the individual might encounter difficulty when attempting to summarize or communicate his or her mental state to others. The problem of understanding and communicating mental states becomes even more difficult when the mental states of multiple individuals are considered.

Gaining insight into the mental states of multiple individuals represents an important tool for understanding events. However, it is also very difficult to properly interpret mental states when the individuals under consideration may themselves be unable to accurately communicate their mental states. Adding to the difficulty is the fact that multiple individuals can have similar or very different mental states when taking part in the same shared activity.

For example, the mental state of two friends can be very different after a certain team wins an important sporting event. Clearly, if one friend is a fan of the winning team, and the other friend is a fan of the losing team, widely varying mental states can be expected. However, the problem of defining the mental states of more than one individual to stimuli more complex than a sports team winning or losing can be a much more difficult exercise in understanding mental states.

Ascertaining and identifying multiple individuals' mental states in response to a common event can provide powerful insight into both the impact of the event and the individuals' mutual interaction and communal response to the event. For example, if a certain television report describing a real-time, nearby, emotionally-charged occurrence is being viewed by a group of individuals at a certain venue and causes a common mental state of concern and unrest, the owner of the venue may take action to alleviate the concern and avoid an unhealthy crowd response. Additionally, when individuals are aware of their mental state(s), they are better equipped to realize their own abilities, cope with the normal stresses of life, work productively and fruitfully, and contribute to their communities.

### SUMMARY

A computer can be used to collect mental state data from an individual, analyze the mental state data, and render an output related to the mental state. Mental state data from a large group of people can be analyzed to identify signatures for certain mental states. Signatures can be automatically clustered and identified using classifiers. The signature can be considered an event definition and can be a function of expression changes among individual(s). A computer-implemented method for analysis is disclosed comprising: obtaining a plurality of videos of people; analyzing the plurality of videos using classifiers; performing expression clustering based on the analyzing; and determining a temporal signature for an event based on the expression clustering. The signature can include a time duration, a peak intensity, a shape for an intensity transition from low intensity to a peak intensity, a shape for an intensity transition from a peak intensity to low intensity, or other components. In some embodiments, the analyzing includes: identifying a human face within a frame of a video selected from the plurality of videos; defining a region of interest (ROI) in the frame that includes the identified human face; extracting one or more histogram-of-oriented-gradients (HoG) features from the ROI; and computing a set of facial metrics based on the one or more HoG features

In embodiments, a computer program product embodied in a non-transitory computer readable medium for analysis can include: code for obtaining a plurality of videos of people; code for analyzing the plurality of videos using classifiers; code for performing expression clustering based on the analyzing; and code for determining a temporal signature for an event based on the expression clustering. Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for mental state event definition generation.

FIG. 2 is a flow diagram for video analysis for a face.

FIG. 3 is a flow diagram for video analysis for multiple faces.

FIG. 4 is a diagram showing cameras obtaining images of a person.

FIG. 5 shows example image collection including multiple mobile devices.

FIG. 6 shows example clustering by parameter.

FIG. 7 shows an example plot for smile peak and duration.

FIG. 8 is an example showing peak rise time of smiles.

FIG. 9 is a flow diagram from a server perspective.

FIG. 10 is a flow diagram from a device perspective.

FIG. 11 is a flow diagram for rendering an inferred mental state.

FIG. 12 shows example facial data collection including landmarks.

FIG. 13 is a flow for detecting facial expressions.

FIG. 14 is a flow for the large-scale clustering of facial events.

FIG. 15 shows example unsupervised clustering of features and characterizations of cluster profiles.

FIG. 16A shows example tags embedded in a webpage.

FIG. 16B shows example of invoking tags to collect images.

FIG. 17 is a system for mental state event definition generation.

### DETAILED DESCRIPTION

People sense and react to external stimuli daily, experiencing those stimuli through their primary senses. The familiar primary senses such as hearing, sight, smell, taste, and touch, along with additional senses such as balance, pain, temperature, and so on, can create certain sensations or feelings and can cause people to react in different ways and experience a range of mental states when exposed to certain stimuli. The experienced mental states can include delight, disgust, calmness, doubt, hesitation, excitement, and many others. External stimuli to which the people react can be naturally generated or human-generated. For example, naturally generated stimuli can include breathtaking views, awe inspiring storms, birdsongs, the smells of a pine forest, the feel of a granite rock face, and so on. Human-generated stimuli can impact the senses and can include music, art, sports events, fine cuisine, and various media such as advertisements, movies, video clips, television programs, etc. The stimuli can include immersive shared social experiences such as shared videos. The stimuli can also be virtual reality or augmented reality videos, images, gaming, or media. People who are experiencing the external stimuli can be monitored to determine their reactions to the stimuli. Reaction data can be gathered and analyzed to discern mental states being experienced by the people. The gathered data can include visual cues, physiological parameters, and so on. Data can be gathered from many people experiencing the same external stimulus, where the external stimulus is an event affecting many people, such as a sporting match or an opera, for example. The people who are encountering the same external stimulus might experience similar mental states. For example, people viewing the same comedic performance can experience happiness and amusement, as evidenced by collective smiling and laughing, among other markers.

In embodiments of the present disclosure, techniques and capabilities for qualifying the reaction of people to a stimulus are described. Continuing the example given above, other comedic performances can be shown and the peoples' reactions to the further performances can be gathered in order to qualify people's reactions to the first comedic performance. Using a plurality of shows and data sets, the happiness and amusement that result from viewing comedy performances can be identified and an event signature—an event definition—can be determined. The event signature can be determined from data gathered on the people experiencing the event and can include lengths of expressions, peak intensities of individual's expressions, a shape for an intensity transition from low intensity to a peak expression intensity, and/or a shape for an intensity transition from a peak intensity to low expression intensity. The signatures can be used to create a taxonomy of expressions. For instance, varying types of smiles can be sorted using categories such as humorous smiles, laughing smiles, sympathetic smiles, sad smiles, melancholy smiles, skeptical smiles, and so on. Once a clear event signature has been generated, queries can be made for expression occurrences and even, in certain examples, for the effectiveness of a joke or other stimulus.

Data gathered on people experiencing an event can further comprise videos collected by a camera. The videos can contain a wide range of useful data such as facial data for the plurality of people. As increasing numbers of videos are collected on the plurality of people experiencing and reacting to a range of different types of events, mental states can be determined and event signatures can begin to emerge for the various event types. As people can experience a range of mental states as a result of experiencing external stimuli and the reactions of different people to the same stimulus can be varied, not all of the people will experience the same mental states for a given event. Specifically, the mental states of individual people can range widely from one person to another. For example, while some people viewing the comedy performance will find it funny and react with amusement, others will find it silly or confusing and react instead with boredom. The mental states experienced by the plurality of people in response to the event can include sadness, stress, anger, happiness, disgust, frustration, confusion, disappointment, hesitation, cognitive overload, focusing, engagement, attention, boredom, exploration, confidence, trust, delight, skepticism, doubt, satisfaction, excitement, laughter, calmness, and curiosity, for example. The particular mental states experienced by the people experiencing an external stimulus can be determined by collecting data from the people, analyzing the data, and inferring mental states from the data.

Emotions and mental states can be determined by examining facial expressions and movements of people experiencing an external stimulus. The Facial Action Coding System (FACS) is one system that can be used to classify and describe facial movements. FACS supports the grouping of facial expressions by the appearance of various muscle movements on the face of a person who is being observed. Changes in facial appearance can result from movements of individual facial muscles. Such muscle movements can be coded using the FACS into various facial expressions. The FACS can be used to extract emotions from observed facial movements, as facial movements can present a physical representation or expression of an individual's emotions. The facial expressions can be deconstructed into Action Units (AU), which are based on the actions of one or more facial muscles. There are many possible AUs, including inner brow raiser, lid tightener, lower lip depressor, wink, eyes turn right, and head turn left, among many others. Temporal segments can also be included in the facial expressions. The temporal segments can include rise time, fall time, rate of rise, rate of fall, duration, and so on, for describing temporal characteristics of the facial expressions. The AUs can be used for recognizing basic emotions. In addition, intensities can be assigned to the AUs. The AU intensities are denoted using letters and range from intensity A (trace), to intensity E (maximum). So, AU 4A denotes a weak trace of AU 4 (brow lowerer), while AU 4E denotes a maximum intensity of expression AU 4 for a given individual.

The mental state analysis used to determine the mental states of people experiencing external stimuli is based on processing video data collected from the group of people. The external stimuli experienced by the people can include viewing a video or some other event. In some embodiments, part of the plurality of people view a different video or videos, while in others the entire plurality views the same video. Video monitoring of the viewers of the video can be performed, where the video monitoring can be active or passive. A wide range of devices can be used for collecting the video data including mobile devices, smartphones, PDAs, tablet computers, wearable computers, laptop computers, desktop computers, and so on, any of which can be fitted with a camera. Other devices can also be used for the video data collection including smart and “intelligent” devices such as Internet-connected devices, wireless digital consumer devices, smart televisions, and so on. The collected data can be analyzed using classifiers, where the classifiers can include expression classifiers. The classifiers can be used to determine the expressions, including facial expressions, of the people who are being monitored. Further, the expressions can be classified based on the analysis of the video data, allowing clustering of the instances of certain expressions to be performed. In turn, the clustered expressions can be used to determine an expression signature. Based on the expression signature, an event definition can be generated. The expression signature can be based on a certain media instance. However, in many embodiments the expression signature is based on many videos being collected and the recognition of certain expressions based on clustering of the expressions. The clustering can be a grouping of similar expressions and the signature can include a time duration and a peak intensity for expressions. In some embodiments, the signature can include a shape showing the transition of the intensity as well. Clustered expressions resulting from the analyzed data can include smiling, smirking, brow furrowing, and so on.

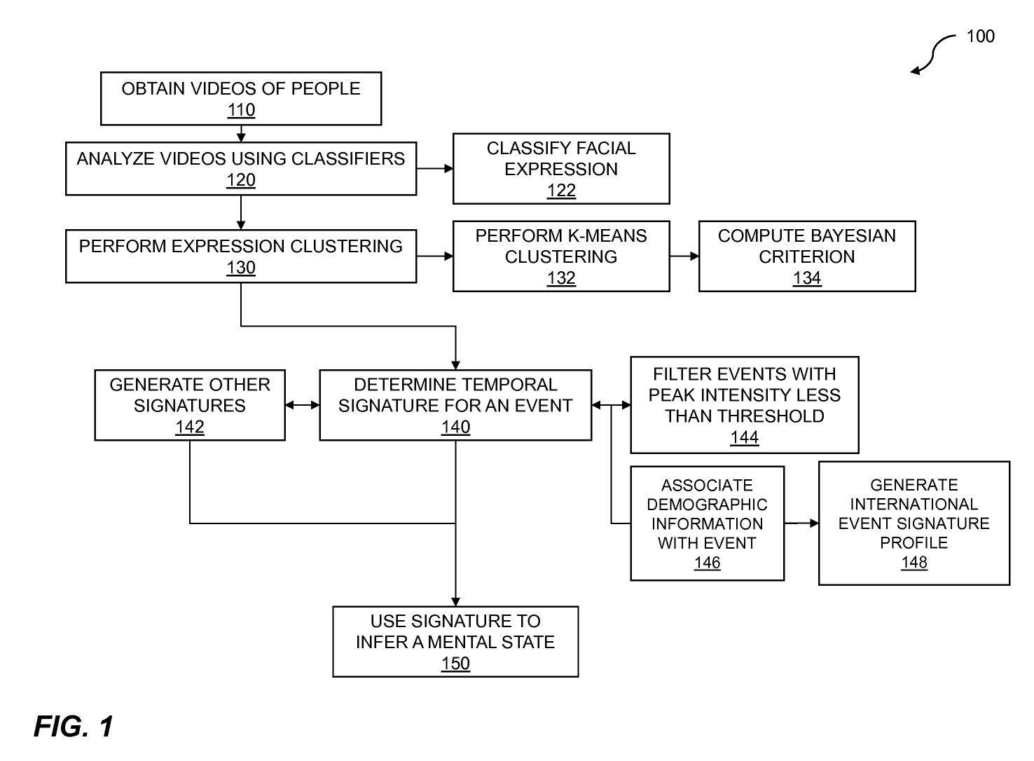

FIG. 1 is a flow diagram for mental state event definition generation. A flow 100 that describes a computer-implemented method for analysis is shown. The flow 100 includes obtaining a plurality of videos of people 110. The plurality of videos which can be obtained can include videos of people engaged in various activities including experiencing various stimuli. The external stimuli can be naturally generated stimuli or man-made stimuli. The stimuli can be experienced by the people through one or more senses, for example. As used herein, senses can include the primary human senses such as hearing, sight, smell, taste, and touch, as well as additional senses such as balance, pain, temperature, and so on. In many embodiments, the stimulus includes experiencing an event. The event can comprise watching a media presentation, for example. The plurality of videos can be of people who are experiencing similar situations or different situations. The videos of the people can be obtained using various types of video capture devices. The video capture devices can include a webcam, a video camera, a still camera, a thermal imager, a CCD device, a camera connected to a digital device such as a smart phone, a three-dimensional camera, a light field camera, multiple cameras working together, and any other type of video capture technique that can allow captured data to be used in an electronic system.

The flow 100 includes analyzing the plurality of videos using classifiers 120. The analyzing can be performed for a variety of purposes including analyzing mental states. The analyzing can be based on one or more classifiers. Any number of classifiers appropriate to the analysis can be used, including a single classifier or a plurality of classifiers, depending on the embodiment. The classifiers can be used to identify a category to which a video belongs, but the classifiers can also place the video in multiple categories, considering that a plurality of categories can be identified. In embodiments, a classifier, from the classifiers, is used on a mobile device where the plurality of videos are obtained using the mobile device. The categories can be various categories appropriate to the analysis. The classifiers can be algorithms and mathematical functions that can categorize the videos, and can be obtained by a variety of techniques. For example, the classifiers can be developed and stored locally, can be purchased from a provider of classifiers, can be downloaded from a web service such as an ftp site, and so on. The classifiers can be categorized and used based on the analysis requirements. In a situation where videos are obtained using a mobile device and classifiers are also executed on the mobile device, the device might require that the analysis be performed quickly while using minimal memory, and thus a simple classifier can be implemented and used for the analysis. Alternatively, a requirement that the analysis be performed accurately and more thoroughly than is possible with only a simple classifier can dictate that a complex classifier be implemented and used for the analysis. Such complex classifiers can include one or more expression classifiers, for example. Other classifiers can also be included.

The flow 100 includes classifying the facial expression 122. In embodiments, multiple facial expression classifications are used. The facial expressions can be categorized by emotions, such as happiness, sadness, shock, surprise, disgust, and/or confusion. In embodiments, metadata is stored with the classification, such as information pertaining to the media the subject was viewing at the time of the facial expression that was classified, the age of the viewer, and the gender of the viewer, to name a few.

The flow 100 includes performing expression clustering 130 based on the analyzing. Expression clustering can be performed for a variety of purposes including mental state analysis. The expression clustering can include a variety of facial expressions and can be for smiles, smirks, brow furrows, squints, lowered eyebrows, raised eyebrows, or attention. The expression clustering can be based on action units (AUs), with any appropriate AUs able to be considered for the expression clustering such as inner brow raiser, outer brow raiser, brow lowerer, upper lid raiser, cheek raiser, lid tightener, lips toward each other, nose wrinkle, upper lid raiser, nasolabial deepener, lip corner puller, sharp lip puller, dimpler, lip corner depressor, lower lip depressor, chin raiser, lip pucker, tongue show, lip stretcher, neck tightener, lip funneler, lip tightener, lips part, jaw drop, mouth stretch, lip suck, jaw thrust, jaw sideways, jaw clencher, [lip] bite, [cheek] blow, cheek puff, cheek suck, tongue bulge, lip wipe, nostril dilator, nostril compressor, glabella lowerer, inner eyebrow lowerer, eyes closed, eyebrow gatherer, blink, wink, head turn left, head turn right, head up, head down, head tilt left, head tilt right, head forward, head thrust forward, head back, head shake up and down, head shake side to side, head upward and to the side, eyes turn left, eyes left, eyes turn right, eyes right, eyes up, eyes down, walleye, cross-eye, upward rolling of eyes, clockwise upward rolling of eyes, counter-clockwise upward rolling of eyes, eyes positioned to look at other person, head and/or eyes look at other person, sniff, speech, swallow, chewing, shoulder shrug, head shake back and forth, head nod up and down, flash, partial flash, shiver/tremble, or fast up-down look. The classifiers can be implemented in such a way that the expression clustering can be based on the analyzing of the videos using the classifiers, but the expression clustering can also be based on self-reporting by the people from whom the videos were obtained, including self-reporting performed by an online survey, a survey app, a web form, a paper form, and so on. The self-reporting can take place immediately following the obtaining of the video of the person, or at another appropriate time, for example.

The flow can include performing K-means clustering 132. In embodiments, the K value is used to define the number of clusters. This in turn can result in K centroids, one for each cluster. The initial placement of the centroids places them, in some embodiments, as far away from each other as possible. Then, each point belonging to a given data set can be associated to the nearest centroid. When no point is pending, the first step is completed and an initial grouping is finished. Then, new centroids are computed based on the clusters resulting from the previous step. Once the K new centroids are derived, a new binding is performed with the same data set points and the nearest new centroid. This process iterates until the centroids do not move anymore and converge upon a final location.

The flow 100 can include computing a Bayesian criterion 134. In embodiments, in order to select the number of clusters, a Bayesian Information Criterion (BICk) for K in 1, 2, . . . , 10 is computed. That is, embodiments include computing a Bayesian information criterion value for a K value ranging from one to ten. The smallest K is then selected where (1−BICk+1/BICk)<0.025. In embodiments, for smile and eyebrow raiser the smallest K corresponds to five clusters and for eyebrow lowerer it corresponds to four clusters.

The flow 100 includes determining a temporal signature for an event 140 based on the expression clustering. An event can be defined as any external stimulus experienced by the people from whom video was collected, for example. The event can include viewing a media presentation, where the media presentation can comprise a video, among other possible media forms. The signature for the event can be based on various statistical, mathematical, or other measures. In particular, the event can be characterized by a change in facial expression over time. Of particular interest are rise and hold times, which pertain to how quickly the facial expression formed, and how long it remained. For example, if someone quickly smiles (e.g. within 500 milliseconds), the rise time can be considered short. Whereas if someone gradually smiles with increasing intensity over several seconds, the rise time is longer. Another measure is how long the person continued with the smile, or another expression of interest, based on the stimulus. The signature can include an emotion, in that the identified signature can show collective or individual emotional response to external stimuli. Any emotion can be included in the signature for the event, including one or more of humor, sadness, poignancy, and mirth. Other emotions such as affection, confidence, depression, euphoria, distrust, hope, hysteria, passion, regret, surprise, and zest can also be included. As previously noted, the signature can include time duration information on facial expressions such as a rise time, a fall time, a peak time, and so on, for various expressions. The signature can also include a peak intensity for expressions. The peak intensity can range from a weakest trace to a maximum intensity as defined by a predetermined scale such as the AU intensity scale. The rating of the intensity can be based on an individual person, on a group of people, and so on. The signature can include a shape for an intensity transition from low intensity to a peak intensity, thus quantifying facial expression transitions as part of the signature. For example, the shape for a low-to-peak intensity transition can indicate a rate at which the transition occurred, whether the peak intensity was sharp or diffuse, and so on. Conversely, the signature can include a shape for an intensity transition from a peak intensity to low intensity as another valuable quantifier of facial expressions. As above, the shape of the peak-to-low intensity transition can indicate a rate at which the transition occurred along with various other useful characteristics relating to the transition. The determining can also include generating other signatures 142 for other events based on the analyzing, or as a result of the analyzing. The other signatures can relate to secondary expressions and can be generated to clarify nuances in a given signature. Returning to the previously mentioned example of a comedic performance, a signature can be determined for a certain type of comedic performance, but in some situations, it might prove helpful to generate further signatures for certain audiences watching a certain instance of the comedic performance. That is, while a plurality of people are watching a comedic performance that has already had a signature defined, a second signature can be generated on the group to define a new subgenre of comedic performance, for example.

The flow 100 may further comprise filtering events having a peak intensity that is below a predetermined threshold 144. In embodiments, expressions are ranked in intensity on a fixed scale, for example from zero to ten, where an intensity value of zero indicates no presence of the desired expression, and an intensity value of ten indicates a maximum presence of the desired expression. In embodiments, the predetermined threshold is a function of a maximum peak intensity. For example, the predetermined threshold can be established as 70 percent of the maximum peak intensity. Thus, if the maximum peak intensity is 90 in an embodiment, then the predetermined threshold would be set to 63. The intensity of the expression can be evaluated based on a variety of factors, such as facial movement, speed of movement, magnitude and direction of movement, to name a few. For example, in a situation where a plurality of faces are being monitored for surprise, the facial features that are evaluated can include a number of brow raises, and, if mouth opens are detected, the width and time duration of the mouth opens. These and other criteria can be used in forming the intensity value. In embodiments, an average intensity value is computed for a group of people. Consider an example where the “shock” effect of a piece of media is being evaluated, such as an episode of a murder mystery show. The creators of the murder mystery show can utilize disclosed embodiments to preview the episode to a group of people. The surprise factor can be evaluated over the course of the episode. In order to identify points in the episode that were perceived to cause surprise, a filter can be applied to ignore any spikes in intensity that fall below a predetermined value. For example, using a scale of zero to ten as previously described, a predetermined threshold value of seven can be chosen, such that only intensity peaks greater than seven are indicated as surprise moments. The intensity peaks that exceed the predetermined threshold can be referred to as “significant events.” The time of the significant events can be correlated with the point in the episode to identify which parts of the episode caused surprise and which parts did not. Such a system enables content creators, such as movie and television show producers, to evaluate how well the episode achieves the content creators' intended effects.

The flow 100 can further comprise associating demographic information with an event 146. The demographic information can include country of residence. The demographic information can also include, but is not limited to, gender, age, race, and level of education. The flow can further include generating an international event signature profile 148. That is, by utilizing country of residence information associated with each person undergoing the expression analysis, it is possible to see how certain events are interpreted across various cultures. For example, the demographic information can be classified by continent. Thus, people from North America, South America, Europe, Asia, and Australia can be shown a piece of media content, and then international event signatures can be computed using the demographic information. Thus, embodiments provide a way to learn how an event is perceived differently by people in different countries and cultures. In some instances, one group can find humorous or surprising content that is off-putting or offensive to another group.

The flow 100 further comprises using the signature to infer a mental state 150. The mental state can include one or more of sadness, stress, anger, happiness, disgust, frustration, confusion, disappointment, hesitation, cognitive overload, focusing, engagement, attention, boredom, exploration, confidence, trust, delight, skepticism, doubt, satisfaction, excitement, laughter, calmness, and curiosity. A mental state can be inferred for a person and for a plurality of people. The mental state can be inferred from the signature for the event. Additional mental states can be inferred from the other signatures generated for the event. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

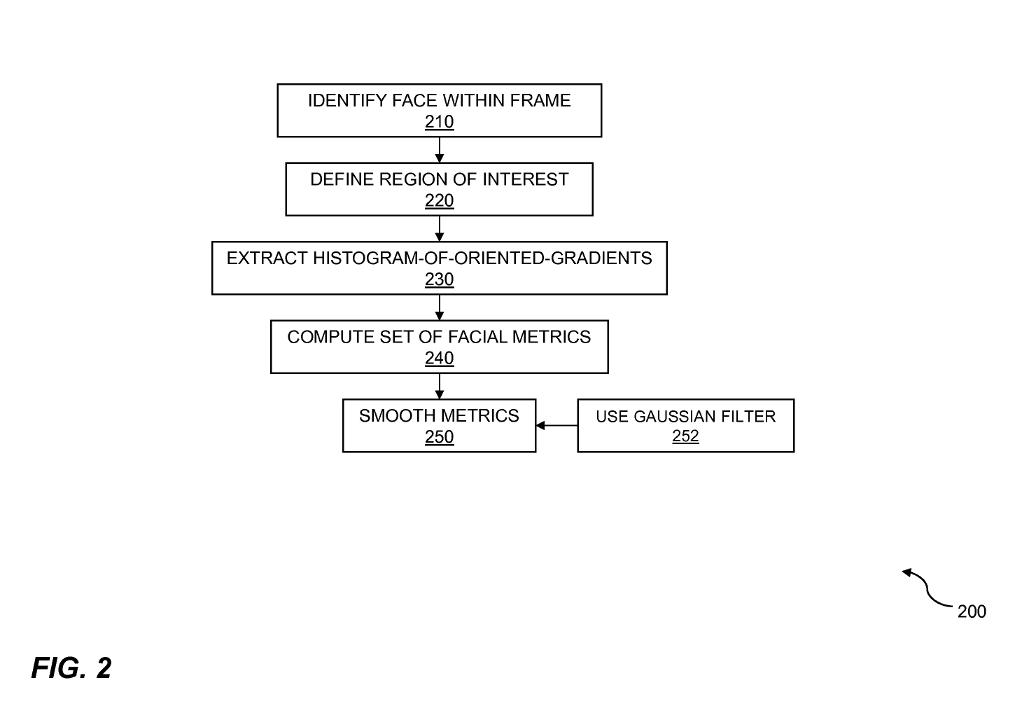

FIG. 2 is a flow diagram 200 for video analysis for a face. Video analysis for a face is used in various embodiments of the present disclosure to track the mental state of one or more people for the purposes of generating mental state data, such as determining a temporal signature for an event and/or generating an international event profile. The flow 200 starts with identifying a face within a frame 210. The frame can be a frame of video. The face can be identified by the use of landmarks, such as eyes, a nose, and a mouth. In embodiments, a Hidden Markov Model (HMM) is used as a recognition algorithm for identifying the face. The flow continues with defining a region of interest 220. The region of interest (ROI) is, in some embodiments, even larger than the full face, while other embodiments, it is smaller than the area of the face. For example, a ROI can include the eyes, nose, and mouth of the face, but might exclude the top of the head and ears. The flow 200 then continues with extracting histogram-of-oriented-gradients (HoGs) 230. Extracting HoGs involves counting occurrences of gradient orientation in the region of interest in order to quantify each cell's edge directions and thus predict object locations within an image. The flow 200 then continues with computing a set of facial metrics 240. In embodiments, a support vector machine (SVM) classifier, with radial basis function (RBF) kernel, is applied to HoG features to compute the set of facial metrics 240. The flow 200 then continues with smoothing the metrics 250. In embodiments, the smoothing 250 is performed using a Gaussian filter 252 (σ=3) to remove high frequency noise and prevent spurious peaks from being detected.

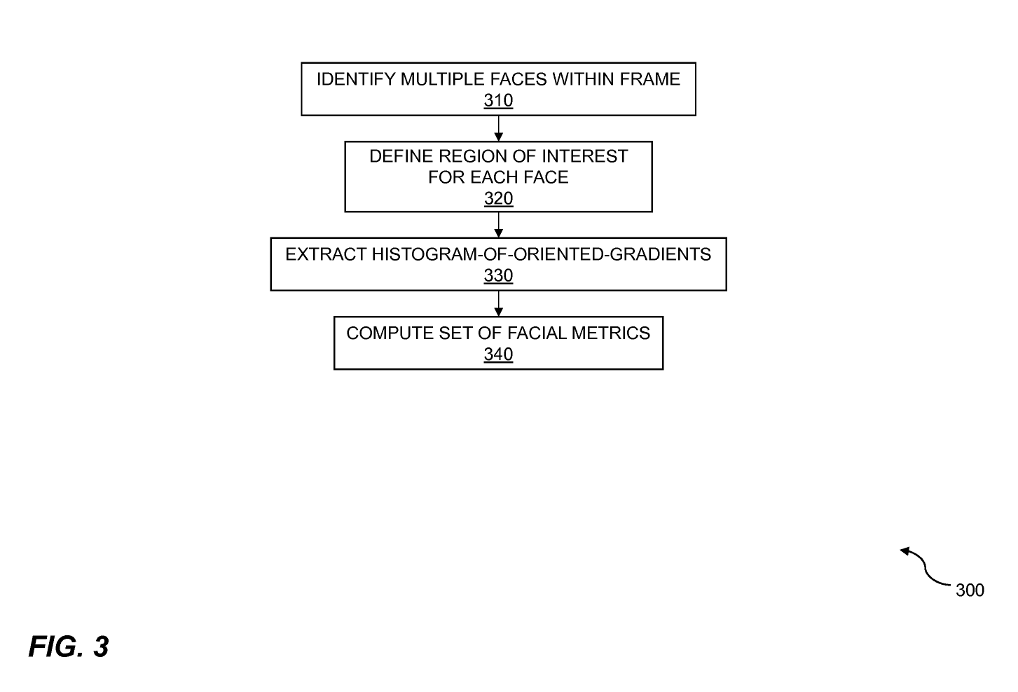

FIG. 3 is a flow diagram 300 for video analysis for multiple faces. It is expedient in some embodiments of the present disclosure to evaluate the mental states of a unified audience. For example, a wide angle camera can be positioned such that it captures the faces of multiple people sitting in a room watching an event. The event can be, for example, a live presentation, a live demonstration, or a pre-recorded event. Each frame can contain multiple faces. In embodiments, the camera can be an infrared camera that can be used in low light conditions, such as in a movie theater, for example. Each frame of such a video will contain multiple faces. In embodiments, the frames contain more than one face, with some embodiments containing more than 200 faces. The flow 300 starts by identifying the multiple faces within a frame 310. The flow 300 continues with defining a region of interest for each face 320. After the defining, a HoG 330 can be extracted for each region of interest. The flow 300 then continues with computing a set of facial metrics 340 for each face that was detected. In this way, multiple faces can be simultaneously analyzed with a single camera. Embodiments can further include smoothing each metric from the set of facial metrics. In some embodiments, the smoothing is performed using a Gaussian filter. Thus, embodiments can include identifying multiple human faces within a frame of a video selected from the plurality of videos; defining a region of interest (ROI) in the frame for each identified human face; extracting one or more HoG features from each ROI; and computing a set of facial metrics based on the one or more HoG features for each of the multiple human faces.

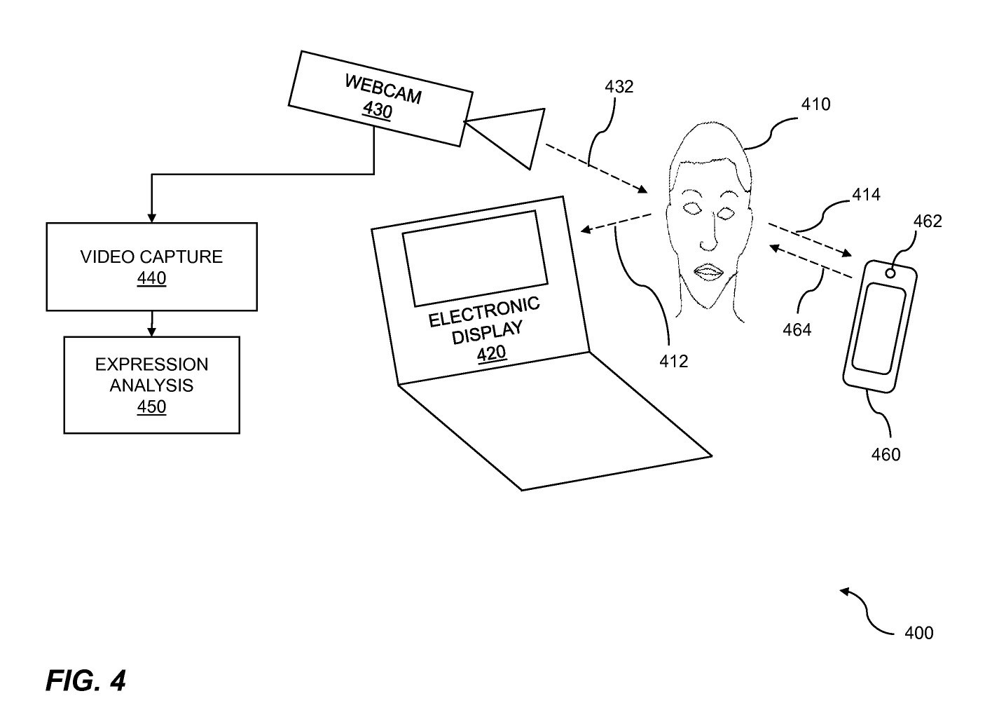

FIG. 4 is a diagram showing cameras obtaining images of a person. The example 400 shows a person 410 viewing an event on one or more electronic displays. In practice, any number of displays can be shown to the person 410. An event can be a media presentation, where the media presentation can be viewed on an electronic display. The media presentation can be an advertisement, a political campaign announcement, a TV show, a movie, a video clip, or any other type of media presentation. In the example 400, the person 410 has a line of sight 412 to an electronic display 420. Similarly, the person 410 has a line of sight 414 to a display of a mobile device 460. While one person has been shown, in practical use, embodiments of the present invention can analyze groups comprising tens, hundreds, or thousands of people or more. In embodiments including groups of people, each person has a line of sight 412 to the event or media presentation rendered on an electronic display 420 and/or each person has a line of sight 414 to the event or media presentation rendered on an electronic display of a mobile device 460. The plurality of captured videos can be of people who are viewing substantially identical media presentations or events, or conversely, the videos can capture people viewing different events or media presentations.

The display 420 can comprise a television monitor, a projector, a computer monitor (including a laptop screen, a tablet screen, a net book screen, and the like), a projection apparatus, and the like. The display 460 can be a cell phone display, a smartphone display, a mobile device display, a tablet display, or another electronic display. A camera can be used to capture images and video of the person 410. In the example 400 shown, a webcam 430 has a line of sight 432 to the person 410. In one embodiment, the webcam 430 is a networked digital camera that can take still and/or moving images of the face and possibly the body of the person 410. The webcam 430 can be used to capture one or more of the facial data and the physiological data. Additionally, the example 400 shows a camera 462 on a mobile device 460 with a line of sight 464 to the person 410. As with the webcam, the camera 462 can be used to capture one or more of the facial data and the physiological data of the person 410.

The webcam 430 can be used to capture data from the person 410. The webcam 430 can be any camera including a camera on a computer (such as a laptop, a net book, a tablet, or the like), a video camera, a still camera, a 3-D camera, a thermal imager, a CCD device, a three-dimensional camera, a light field camera, multiple webcams used to show different views of the viewers, or any other type of image capture apparatus that allows captured image data to be used in an electronic system. In addition, the webcam can be a cell phone camera, a mobile device camera (including, but not limited to, a forward facing camera), and so on. The webcam 430 can capture a video or a plurality of videos of the person or persons viewing the event or situation. The plurality of videos can be captured of people who are viewing substantially identical situations, such as viewing media presentations or events. The videos can be captured by a single camera, an array of cameras, randomly placed cameras, a mix of types of cameras, and so on. As mentioned above, media presentations can comprise an advertisement, a political campaign announcement, a TV show, a movie, a video clip, or any other type of media presentation. The media can be oriented toward an emotion. For example, the media can include comedic material to evoke happiness, tragic material to evoke sorrow, and so on.

The facial data from the webcam 430 is received by a video capture module 440 which can decompress the video into a raw format from a compressed format such as H.264, MPEG-2, or the like. Facial data that is received can be received in the form of a plurality of videos, with the possibility of the plurality of videos coming from a plurality of devices. The plurality of videos can be of one person and of a plurality of people who are viewing substantially identical situations or substantially different situations. The substantially identical situations can include viewing media, listening to audio-only media, and/or viewing still photographs. The facial data can include information on action units, head gestures, eye movements, muscle movements, expressions, smiles, and the like.

The raw video data can then be processed for expression analysis 450. The processing can include analysis of expression data, action units, gestures, mental states, and so on. Facial data as contained in the raw video data can include information on one or more of action units, head gestures, smiles, brow furrows, squints, lowered eyebrows, raised eyebrows, attention, and the like. The action units can be used to identify smiles, frowns, and other facial indicators of expressions. Gestures can also be identified, and can include a head tilt to the side, a forward lean, a smile, a frown, as well as many other gestures. Other types of data including physiological data can be obtained, where the physiological data is obtained through the webcam 430 without contacting the person or persons. Respiration, heart rate, heart rate variability, perspiration, temperature, and other physiological indicators of mental state can be determined by analyzing the images and video data.

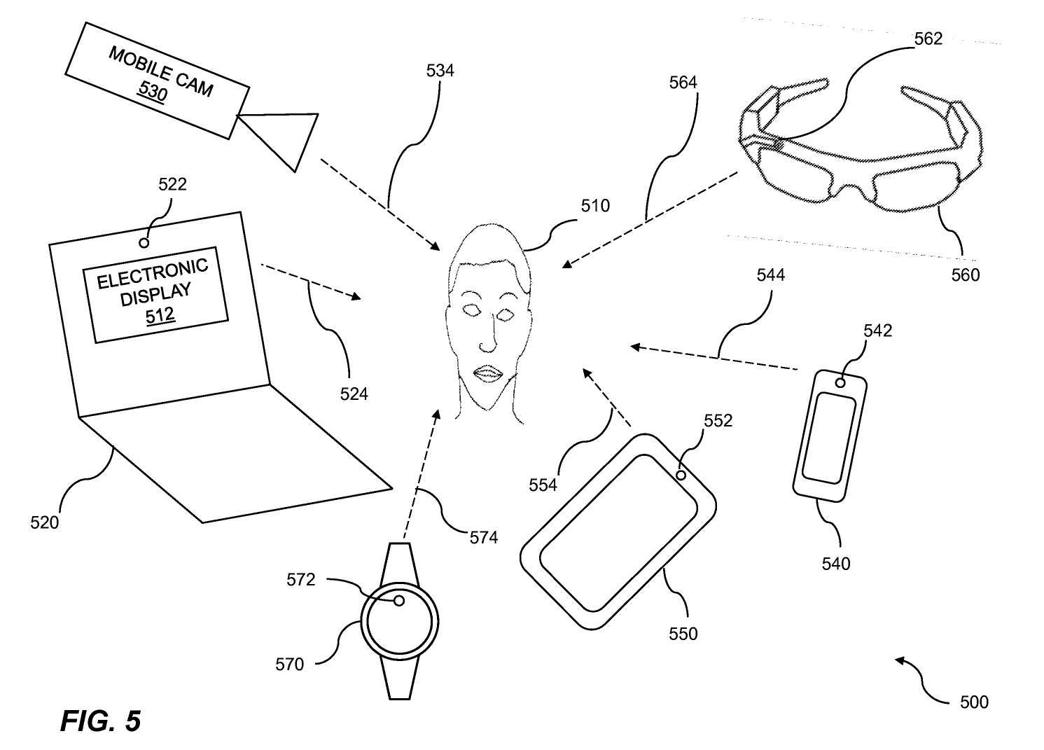

FIG. 5 shows example image collection including multiple mobile devices 500. The multiple mobile devices can be used to collect video data on a person. While one person is shown, in practice the video data on any number of people can be collected. A user 510 can be observed as she or he is performing a task, experiencing an event, viewing a media presentation, and so on. The user 510 can be shown one or more media presentations, for example, or another form of displayed media. The one or more media presentations can be shown to a plurality of people instead of an individual user. The media presentations can be displayed on an electronic display 512. The data collected on the user 510 or on a plurality of users can be in the form of one or more videos. The plurality of videos can be of people who are experiencing different situations. Some example situations can include the user or plurality of users being exposed to TV programs, movies, video clips, and other such media. The situations could also include exposure to media such as advertisements, political messages, news programs, and so on. As noted before, video data can be collected on one or more users in substantially identical or different situations who are viewing either a single media presentation or a plurality of presentations. The data collected on the user 510 can be analyzed and viewed for a variety of purposes including expression analysis. The electronic display 512 can be on a laptop computer 520 as shown, a tablet computer 550, a cell phone 540, a television, a mobile monitor, or any other type of electronic device. In a certain embodiment, expression data is collected on a mobile device such as a cell phone 540, a tablet computer 550, a laptop computer 520, or a watch 570. Thus, the multiple sources can include at least one mobile device such as a phone 540 or a tablet 550, or a wearable device such as a watch 570 or glasses 560. A mobile device can include a forward facing camera and/or a rear-facing camera that can be used to collect expression data. Sources of expression data can include a webcam 522, a phone camera 542, a tablet camera 552, a wearable camera 562, and a mobile camera 530. A wearable camera can comprise various camera devices such as the watch camera 572.

As the user 510 is monitored, the user 510 might move due to the nature of the task, boredom, discomfort, distractions, or for another reason. As the user moves, the camera with a view of the user's face can change. Thus, as an example, if the user 510 is looking in a first direction, the line of sight 524 from the webcam 522 is able to observe the individual's face but if the user is looking in a second direction, the line of sight 534 from the mobile camera 530 is able to observe the individual's face. Further, in other embodiments, if the user is looking in a third direction, the line of sight 544 from the phone camera 542 is able to observe the individual's face, and if the user is looking in a fourth direction, the line of sight 554 from the tablet camera 552 is able to observe the individual's face. If the user is looking in a fifth direction, the line of sight 564 from the wearable camera 562, which can be a device such as the glasses 560 shown and can be worn by another user or an observer, is able to observe the individual's face. If the user is looking in a sixth direction, the line of sight 574 from the wearable watch-type device 570 with a camera 572 included on the device, is able to observe the individual's face. In other embodiments, the wearable device is a another device, such as an earpiece with a camera, a helmet or hat with a camera, a clip-on camera attached to clothing, or any other type of wearable device with a camera or other sensor for collecting expression data. The user 510 can also employ a wearable device including a camera for gathering contextual information and/or collecting expression data on other users. Because the individual 510 can move her or his head, the facial data can be collected intermittently when the individual is looking in a direction of a camera. In some cases, multiple people are included in the view from one or more cameras, and some embodiments include filtering out faces of one or more other people to determine whether the user 510 is looking toward a camera. All or some of the expression data can be continuously or sporadically available from these various devices and other devices.

The captured video data can include facial expressions, and can be analyzed on a computing device such as the video capture device or on another separate device. The analysis of the video data can include the use of a classifier. For example, the video data can be captured using one of the mobile devices discussed above and sent to a server or another computing device for analysis. However, the captured video data including expressions can also be analyzed on the device which performed the capturing. For example, the analysis can be performed on a mobile device where the videos were obtained with the mobile device and wherein the mobile device includes one or more of a laptop computer, a tablet, a PDA, a smartphone, a wearable device, and so on. In another embodiment, the analyzing can comprise using a classifier on a server or other computing device other than the capturing device.

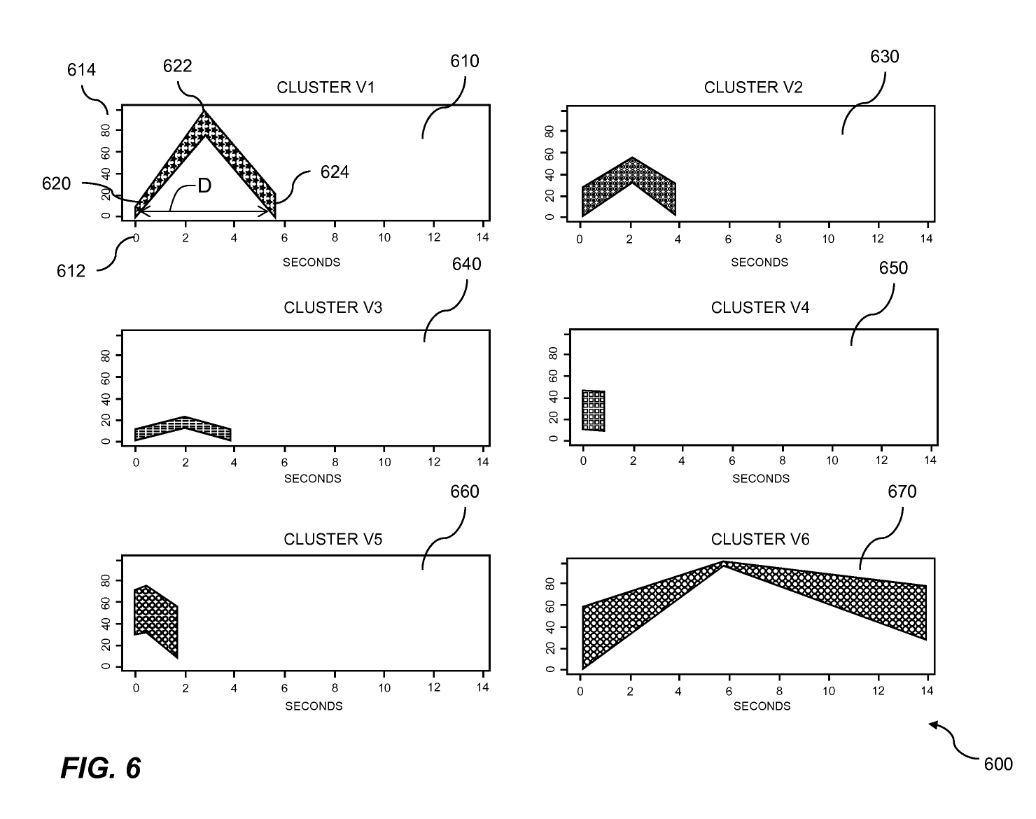

FIG. 6 shows example expression clustering by parameter. In the example graphs 600, smile intensities are shown to illustrate changes and therefore possible components of expression signatures. A component can be a peak intensity value, a difference between a trough and a peak value, a rate of expression change rising towards the peak or descending from the peak, a duration of intensity, and so on. In embodiments, the following signature attributes are tracked: Event Height (maximum value), Event Length (duration between onset and offset), Event Rise (increase from onset to peak), Event Decay (decrease from peak to next offset), Rise Speed (gradient of event rise), and Decay Speed (gradient of event decay). Signature attributes can be used to determine if a significant event occurred and to help determine the intensity and duration of the event.

As described in flows 200 and 300, video data can be obtained and analyzed for expressions, with methods provided to cluster the expressions together based on various factors such as type of expression, duration, and intensity. The expression clusters can be plotted. The various plots in 600 illustrate key information about one or more expression clusters including a peak value of the expression, the length of the peak value, peak rise and decay, peak rise and decay speed, and so on. Further, based on the clustered expressions, a signature can be determined for the event that occurred while video data was being captured for the plurality of people.

A plot 610 is an example plot of an expression cluster (facial expression probability curve). The facial expression probability curve can be used as a signature. The expression clustering can result from the analysis of video data on a plurality of people based on classifiers, as previously noted. The expression clustering can be for smiles, smirks, brow furrows, squints, lowered eyebrows, raised eyebrows, attention, and so on. The expression clustering can be for a combination of facial expressions. The expression cluster plot 610 can include a time scale 612 and a peak value scale 614, where the time scale can be used to determine a duration, and the peak value scale can be used to determine an intensity for a given expression. The intensity can be based on a numeric scale (e.g. 0-10, or 0-100). In the case of smiles, more exaggerated smile features (for example the amount of lip corner raising that takes place during the smile) can result in a higher intensity value. Analysis of the expression cluster can produce a signature for the event that led to the expression cluster. The signature can include a rise rate, a peak intensity, and a decay rate, for example. The signature can include a time duration. For example, the time duration of the signature determined from the expression plot 610 is the difference in time D between the point 620 and the point 624 on the x-axis of the plot 610. The point 620 and the point 624 represent adjacent local minima of a facial expression probability curve. Thus, in embodiments, the length of the signature is computed based on detection of adjacent local minima of a facial expression probability curve. The signature can include a peak intensity. For example, the peak intensity of the plot 610 is represented by the point 622, which in this case is a peak value for an expression occurrence. The point 622 can indicate a peak intensity for a smile, a smirk, and so on. In embodiments, a higher peak value for the point 622 indicates a more intense expression in the plot 610, while a lower value for the point 622 indicates a less intense expression value. A difference between a trough intensity value 620 and a peak intensity value 622, as shown in the y-axis peak value scale 614 of the plot 610, can be a component in a signature. The rate of transition from the point 620 to the point 622, and again from the point 622 to the point 624 can be a component of the signature, and can help define a shape for an intensity transition from a low intensity to a peak intensity. Additionally, the signature can include a shape for an intensity transition from a peak intensity to a low intensity. The shape of the intensity transition can vary based on the event which is viewed by the people and the type of facial expression and associated mental state that is occurring. The shape of the intensity transition can vary based on whether the people are experiencing different situations or whether the people are experiencing substantially identical situations. Further, the signature can include a peak intensity and a rise rate to the peak intensity. The rise rate to the peak intensity can indicate a speed for the onset of an expression. The signature can include a peak intensity and a decay rate from the peak intensity, where the decay rate can indicate a speed for the fade of an expression.

Differing clusters are shown in the other plots within FIG. 6. The plot 670 shows an expression that grows significantly in intensity over a long period of time. The plot 670 also shows an end expression value that has a higher intensity than the starting value. Within the cluster 670, the time period to reach an ending value for the expression represents a significant length. Additionally the peak intensity is shown to be very high and approximately the same for all participants in the data cluster 670, but the beginning values are shown to be widely variant, resulting in a large variance in the expression intensity that can occur for this case of clustering. In embodiments, the plot 670 illustrates an instance where a plurality of people with various states of facial activity moved synchronously towards a smile expression and maintained the smile expression for a significant time period. Thus, the signature depicted in the plot 670 can be indicative of an emotional response that gradually builds up over time. Such a response can occur, for example, when listening to a slowly developing humorous story.

Another plot 630 shows a rather uniform change from a trough value to a peak intensity value. The return to a trough value is achieved in roughly the same time as the time to reach a peak intensity. Thus, the signature depicted in the plot 640 can be indicative of an emotional response that quickly occurs and then dissipates. Such a response can occur, for example, when listening to a fairly serious story with a mildly humorous joke unexpectedly interjected.

Still a different plot 640 shows a small change in intensity and a short duration. Some studies indicate that this type of smile is frequently encountered in south-east Asia and the surrounding areas. In this example the plot 640 can indicate a quick and subtle smile. Yet other plots 650 and 660 show other possible clusters of smiles.

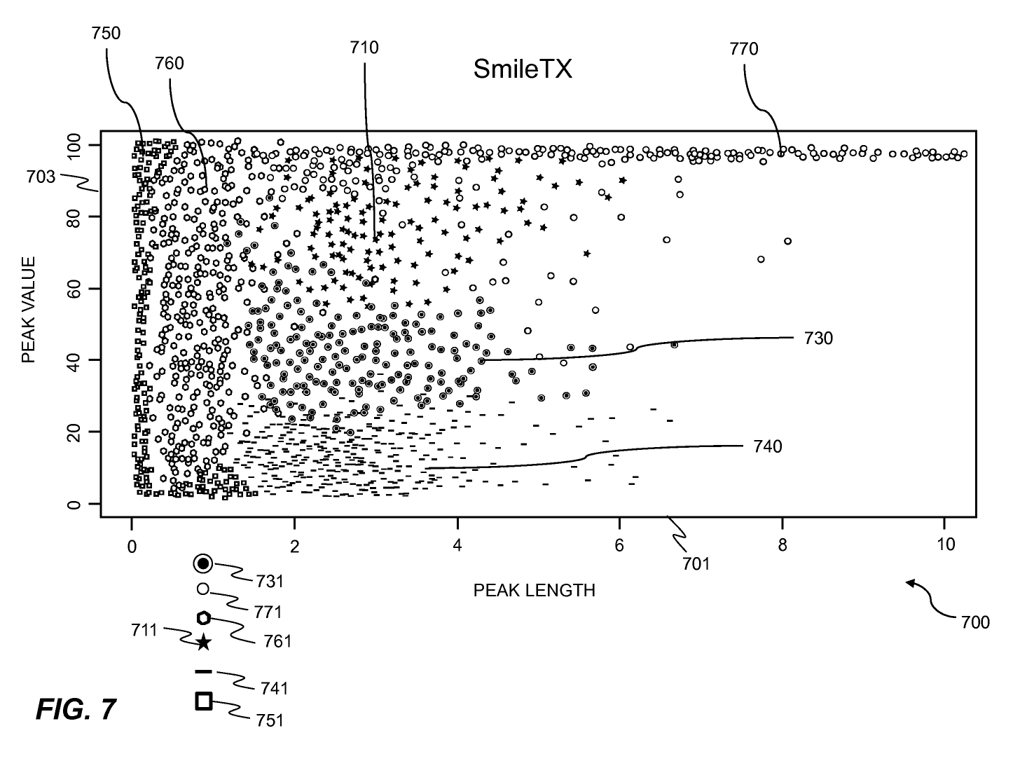

FIG. 7 shows an example plot for smile peak and duration. A plot 700 can be made showing a scatter of expression data resulting from the analyzing of a plurality of videos using classifiers. In this figure, the plotted expression data includes data for six different events. The event data legend symbols are indicated by the symbols 711, 731, 741, 751, 761, and 771, respectively. Each set of event data corresponds to a plot in FIG. 6. Data pertaining to the symbol 711 is associated with the plot 610 of FIG. 6. Data pertaining to the symbol 731 is associated with the plot 630 of FIG. 6. Data pertaining to the symbol 741 is associated with the plot 640 of FIG. 6. Data pertaining to the symbol 751 is associated with the plot 650 of FIG. 6. Data pertaining to the symbol 761 is associated with the plot 660 of FIG. 6. Data pertaining to the symbol 771 is associated with the plot 670 of FIG. 6. The plot 700 shows smile peak duration versus smile peak value. The data point 710 is a representative data point associated with the plot 610 of FIG. 6. The data point 730 is a representative data point associated with the plot 630 of FIG. 6. The data point 740 is a representative data point associated with the plot 640 of FIG. 6. The data point 750 is a representative data point associated with the plot 650 of FIG. 6. The data point 760 is a representative data point associated with the plot 660 of FIG. 6. The data point 770 is a representative data point associated with the plot 670 of FIG. 6. The horizontal axis 701 of the plot 700 represents time in seconds. The vertical axis 703 of the plot 700 represents an intensity value, ranging from a minimum intensity of zero to a maximum intensity of 100. Thus, the plot 700 of FIG. 7 shows a temporal relationship of the intensity of an event signature.

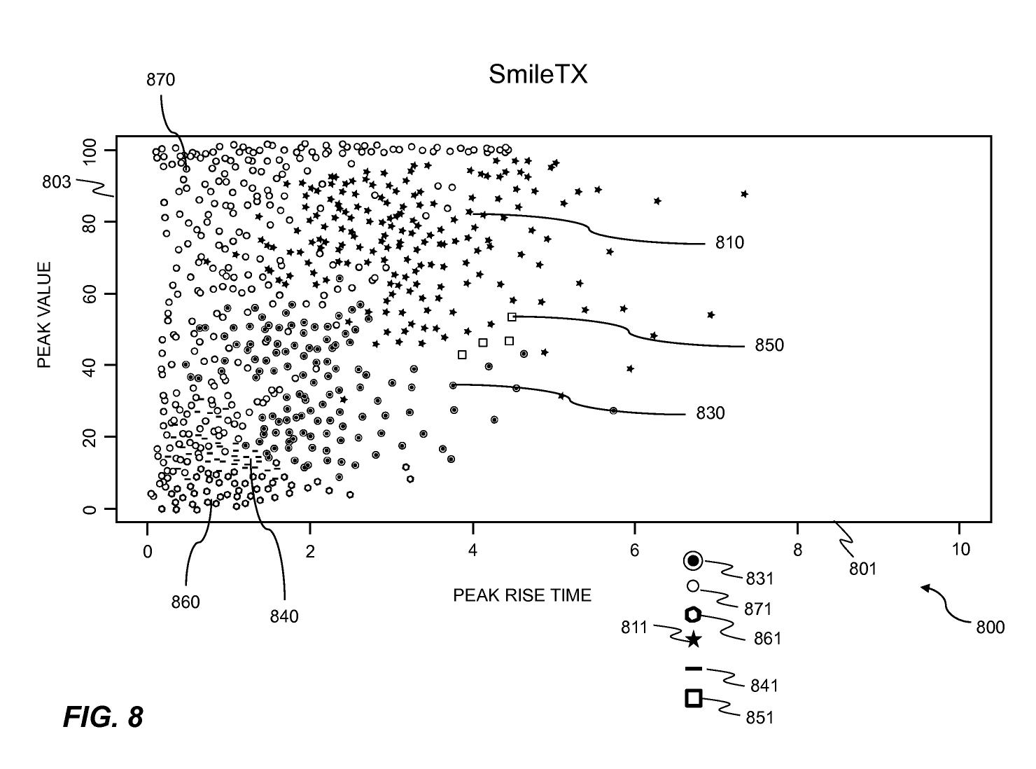

FIG. 8 is an example showing peak rise time of smiles. The example 800 illustrates another way of visualizing the data given in FIG. 6. The information shown in example 800 is a derivative of the temporal relationship of the intensity of an event signature. That is, the example 800 shows the rate of change in expressions over time. A plot can be made which shows rise speed and peak intensity for an expression. The rise speed will display an onset rate for an expression.

The event data legend symbols are indicated by the symbols 811, 831, 841, 851, 861, and 871. Each set of event data corresponds to a plot in FIG. 6. Data pertaining to the symbol 811 is associated with the plot 610 of FIG. 6. Data pertaining to the symbol 831 is associated with the plot 630 of FIG. 6. Data pertaining to the symbol 841 is associated with the plot 640 of FIG. 6. Data pertaining to the symbol 851 is associated with the plot 650 of FIG. 6. Data pertaining to the symbol 861 is associated with the plot 660 of FIG. 6. Data pertaining to the symbol 871 is associated with the plot 670 of FIG. 6. The plot 800 shows peak rise time versus peak rise for smiles. The data point 810 is a representative data point associated with the plot 610 of FIG. 6. The data point 830 is a representative data point associated with the plot 630 of FIG. 6. The data point 840 is a representative data point associated with the plot 640 of FIG. 6. The data point 850 is a representative data point associated with the plot 650 of FIG. 6. The data point 860 is a representative data point associated with the plot 660 of FIG. 6. The data point 870 is a representative data point associated with the plot 670 of FIG. 6. The horizontal axis 801 of the plot 800 represents time in seconds. The vertical axis 803 of the plot 800 represents an intensity value, ranging from a minimum intensity of zero to a maximum intensity of 100.

In practice, any expression could be plotted for peak rise time versus peak rise, where the expressions can include smiles, smirks, brow furrows, squints, lowered eyebrows, raised eyebrows, attention, and so on. The plot can be used, among other things, to show the effectiveness of an event experienced by a plurality of viewers. In particular, the measure of rise speed can be indicative of a measure of surprise, or a rapid transition of emotional states. For example, in terms of comedic material, a fast peak rise can indicate that a joke was funny, and that it was quickly understood. In the case of dramatic material, a rapid transition to a mental state of surprise or sadness can indicate an unexpected twist in a story.

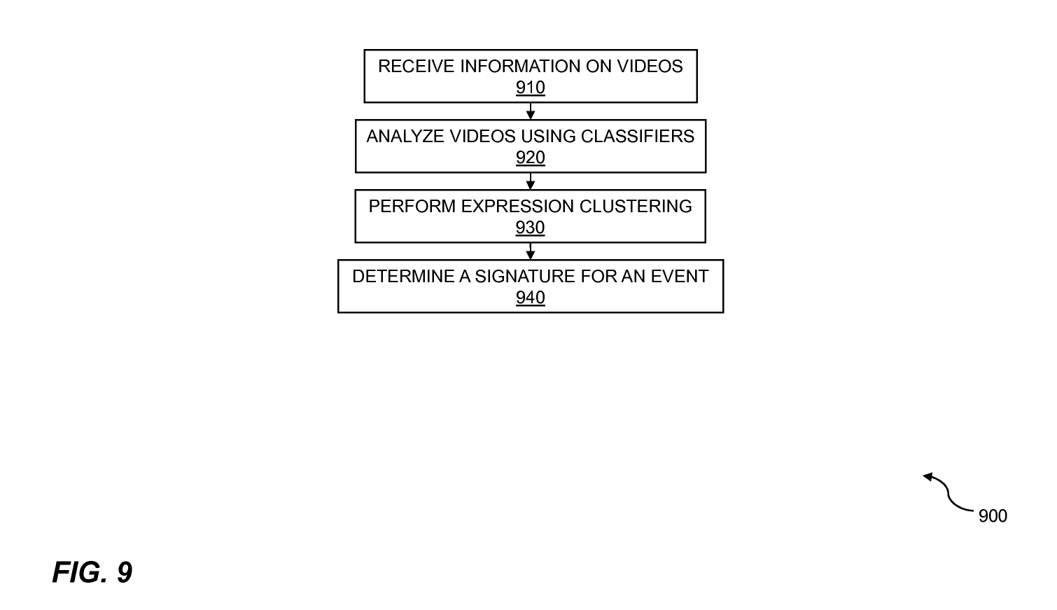

FIG. 9 is a flow diagram from a server perspective. A flow 900 describes a computer-implemented method for analysis from a server perspective. The server can be used to process video data for the purposes of determining a signature for an event. The flow 900 includes receiving information on a plurality of videos of people 910. In some embodiments, the information includes information on the stimulus material (e.g. media being viewed by the people undergoing expression analysis), such as timestamps and scenes within an associated episode. For example, in a 30 minute comedy show, the stimulus material information can include the following:

| Time | Instance | Description |
|---|---|---|
| 2:34 | Joke01 | George states that he is not hungry |
| 5:01 | Antic01 | Elaine begins to dance |
| 7:15 | Antic02 | Jerry gets a pie in the face |
| 24:07 | Surprise01 | Susan dies from an allergic reaction |

In such an embodiment, the video data, along with associated stimulus material information, can be stored in a database where each record in the database includes a time field, an instance field and a description field. When the episode is then viewed by a plurality of people, the mental state information can be correlated to the instances stored in the database. For example, if an event (signature) such as is shown in the plot 610 occurred in the episode around time 5:07, the event correlates to a time shortly after Antic01. This can serve as an indication that the audience reacted considerably to Antic01. Conversely, if a signature such as the one shown in the plot 640 occurred at around time 7:18, that signature correlates to the instance of Antic02. This can indicate a relatively subdued reaction to Antic02. Additionally, if a predetermined threshold of 60 is set as a threshold value for filtering, then responses that do not exceed an intensity of 60 are not counted as events, and are filtered out. With such a filtering scheme, the event corresponding to Antic01 is not filtered, since its peak intensity reaches 100 (see plot 610), whereas the event corresponding to Antic02 is filtered, since it does not exceed the predetermined threshold of 60 (see plot 640). In some embodiments, a correlation window is established to correlate mental state events with the stimulus material. For example, if an event occurs at a time T, then a computer implemented algorithm can search the stimulus material for any instances occurring within a timeframe of (T-X) to T, where X is specified in seconds. Using the example of Antic01 as the event and a value for X of 10 seconds, then when an event occurs at time 5:07 (e.g. the event depicted in the plot 610 of FIG. 6), the algorithm searches the stimulus material for instances from 4:57 to 5:07, which is the correlation window. Based on the example data, Antic01, occurring at time 5:01, falls within the correlation window. Hence, Antic01 is associated with the signature depicted in the plot 610.

The information which is received can include video data on the plurality of people as the people experience an event. The information which is received can further include information on the stimulus material, including occurrence time for specific instances within the stimulus material (e.g. particular jokes, antics, etc.). As mentioned above, the event can include watching a media presentation or being exposed to some other stimulus. The video of the people can be obtained from any video capture device including a webcam, a video camera, a still camera, a light field camera, etc. In some embodiments, an infrared camera can be used, along with an infrared light source, to allow mental state analysis in a low light setting, such as a movie theater, music concert, comedy show, or the like. The information on the plurality of videos of the people can be received via wired and wireless communication techniques. For example, the video data can be received via cellular and PSTN telephony, WiFi, Bluetooth™, Ethernet, ZigBee™, and so on. The received information on the plurality of videos can be stored on the server and by any other appropriate storage technique, including, but not limited to, cloud storage.

The flow 900 includes analyzing the plurality of videos using classifiers 920. The classifiers can be used to identify a category into which the video data can be binned. The analyzing can further comprise classifying a facial expression as belonging to a category of either posed or spontaneous expressions. In some embodiments, the analyzing includes identifying a human face within a frame of a video selected from the plurality of videos; defining a region of interest (ROI) in the frame that includes the identified human face; extracting one or more histogram-of-oriented-gradients (HoG) features from the ROI; and computing a set of facial metrics based on the one or more HoG features. The categories into which the video data can be binned can include facial expressions, for example. A device performing the analysis can include a server, a blade server, a desktop computer, a cloud server, or another appropriate electronic device. The device can use the classifiers for the analyzing. The classifiers can be stored on the device performing the analysis, loaded into the device, provided by a user of the device, and so on. The classifiers can be obtained by wired and wireless communications techniques. The results of the analysis can be stored on the server and by any other appropriate storage technique.

In embodiments, the classifiers can be trained on hand-coded data. An inter-coder agreement of 50% can be used to determine a positive example to be used for training, and 100% agreement on the absence can be used as a criterion for determining a negative example.

The flow 900 includes performing expression clustering based on the analyzing 930. The clustering techniques can include, but are not limited to, K-means clustering, other centroid-based clustering, distribution-based clustering, and/or density-based clustering. The expressions which are used for the expression clustering can include facial expressions, where the facial expressions can include smiles, smirks, brow furrows, squints, lowered eyebrows, raised eyebrows, attention, etc. The expressions which are used for the expression clustering can also include inner brow raiser, outer brow raiser, brow lowerer, upper lid raiser, cheek raiser, lid tightener, and lips toward each other, among many others. The results of the expression clustering can be stored on the server as well as by any other appropriate storage technique.

The flow 900 includes determining a signature for an event 940 based on the expression clustering. The signature which is determined can be based on a number of criteria including a time duration of a peak, an intensity of a peak, and a shape of a transition of an intensity from a low intensity to a peak intensity or from a peak intensity to a low intensity, and so on. A signature can be based on a plot of an expression cluster. The signature can be tied to a type of event, where the event can include viewing a media presentation. The media presentation can include a movie trailer, for example. The signature can be used to infer a mental state, where the mental state can include one or more of sadness, stress, anger, happiness, and so on. The signature which is determined can be stored on the server or by any other appropriate storage technique. Various steps in the flow 900 may be changed in order, repeated, omitted, or the like without departing from the disclosed concepts. Various embodiments of the flow 900 may be included in a computer program product embodied in a non-transitory computer readable medium that includes code executable by one or more processors. In some embodiments, a Hadoop framework can be used to implement a distributed processing system for performing one or more steps of the flow 900.

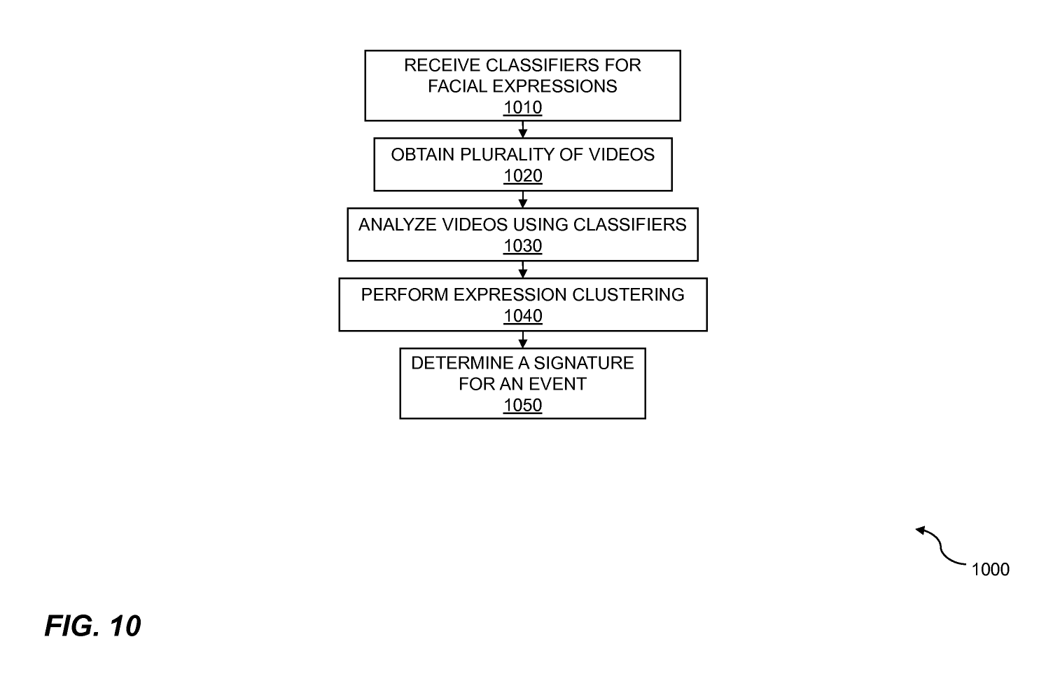

FIG. 10 is a flow diagram from a device perspective. A flow 1000 describes a computer-implemented method for expression analysis from a device perspective. The device can be used both to obtain a plurality of videos of people, and to process the plurality of videos for the purposes of determining a signature for an event. The device can be a mobile device, and can include a laptop computer, a tablet computer, a smartphone, a PDA, a wearable computer, and so on. The flow 1000 includes receiving classifiers for facial expressions 1010. The classifiers can be stored on the mobile device, entered into the mobile device by a user of the mobile device, received using wired and wireless techniques, and so on. The classifiers can be small and/or simple enough to be used within the computational restrictions of the device, where the computational restrictions of the device can include processing power, storage size, etc.

The flow 1000 further includes obtaining a plurality of videos of people 1020. The videos which are obtained can include video data on the plurality of people as the people experience an event. The people can experience the event by viewing the event on an electronic display, and the event can include watching a media presentation. The video of the people can be obtained from any mobile video capture device including a webcam attached to a laptop computer, a camera on a tablet or smart phone, a camera on a wearable device, etc. The obtained videos on the plurality of people can be stored on the mobile device.

The flow 1000 includes analyzing the plurality of videos using the classifiers 1030. The device performing the analysis can use the classifiers to identify a category into which the video data can be binned. The categories into which the video data can be binned can include a category for facial expressions, for example. The facial expressions can include smiles, smirks, squints, and so on. The classifiers can be stored on the device performing the analysis, loaded into the device, provided by a user of the device, and so on. The results of the analysis can be stored on the device.

The flow 1000 includes performing expression clustering 1040 based on the analyzing. The expression clustering can be based on the analysis of the plurality of videos of people. The expressions which are used for the expression clustering can include facial expressions, where the facial expressions can include smiles, smirks, brow furrows, squints, lowered eyebrows, raised eyebrows, attention, and so on. The expressions which are used for the expression clustering also can include inner brow raiser, outer brow raiser, brow lowerer, upper lid raiser, cheek raiser, lid tightener, and lips toward each other, among many others. The results of the expression clustering can be stored on the device.

The flow 1000 includes determining a signature for an event 1050 based on the expression clustering. As was the case for the server-based system, the signature which is determined can be based on a number of criteria including a time duration of a peak, an intensity of a peak, a shape of a transition of an intensity from a low intensity to a peak intensity or from a peak intensity to a low intensity, and so on. The signature can be tied to a type of event, where the event can include viewing a media presentation. The media presentation can include a movie trailer, for example. The signature can be used to infer a mental state, where the mental state can include one or more of sadness, stress, anger, happiness, and so on. The signature which is determined can be stored on the device. Various steps in the flow 1000 may be changed in order, repeated, omitted, or the like without departing from the disclosed concepts. Various embodiments of the flow 1000 may be included in a computer program product embodied in a non-transitory computer readable medium that includes code executable by one or more processors.

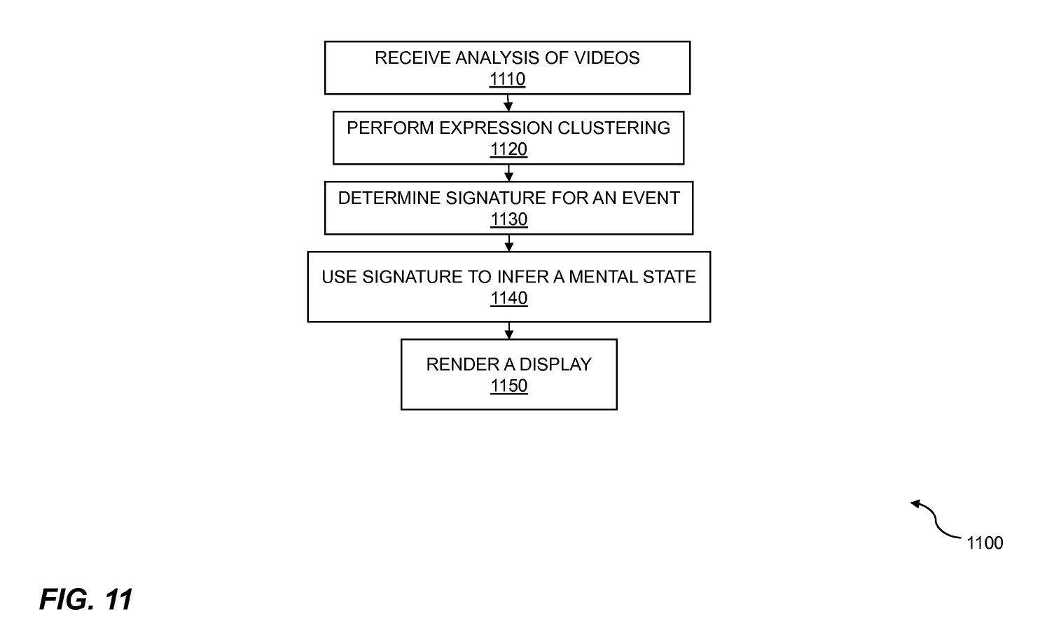

FIG. 11 is a flow diagram for rendering an inferred mental state. A flow 1100 describes a computer-implemented method for analysis and rendering of a mental state. The analysis and rendering can be performed on any appropriate device including a server, a desktop computer, a laptop computer, a tablet, a smartphone, a PDA, a wearable computer, and so on. The device which performs the analysis and the rendering can be used to process the plurality of videos for the purposes of determining a signature for an event as well as to render the signatures and other analysis results on a display. The display can be any type of electronic display, including a television monitor, a projector, a computer monitor (including a laptop screen, a tablet screen, a net book screen, etc.), a projection apparatus, and the like. The display can be a cell phone display, a smartphone display, a mobile device display, a tablet display, or another electronic display. The flow 1100 includes receiving analysis of a plurality of videos of people 1110. The analysis data can be stored in the analysis device, read into the analysis device, entered by the user of the analysis device and so on.

The flow 1100 includes performing expression clustering 1120 based on the analyzing. The expression clustering can be based on the analysis of the plurality of videos of people. The expressions which are used for the expression clustering can include facial expressions. The facial expressions for the clustering can include smiles, smirks, brow furrows, squints, lowered eyebrows, raised eyebrows, attention, and so on. The expression clustering can also include various facial expressions and head gestures. The results of the expression clustering can be stored on the device for later rendering, for further analysis, etc.

The flow 1100 includes determining a signature for an event 1130. The determining of the signature can be based on the expression clustering. As previously discussed, the signature which is determined can be based on a number of criteria including a time duration of a peak, an intensity of a peak, a shape of a transition of an intensity from a low intensity to a peak intensity or from a peak intensity to a low intensity, and so on. The signature can be tied to a type of event, where the event can include viewing a media presentation. The media presentation can include a movie trailer, advertisement, and/or instructional video, to name a few.

The flow 1100 includes using a signature to infer a mental state 1140. The mental state can be the mental state of an individual, or it can be a mental state shared by a plurality of people. The mental state or mental states can result from the person or people experiencing an event or situation. The situation can include a media presentation. The media presentation can include TV programs, movies, video clips, and other such media, for example. The mental states which can be inferred can include one or more of sadness, stress, anger, happiness, and so on. The signature which is determined can be stored on the device for further analysis, signature determination, rendering, and so on.

The flow 1100 includes rendering a display 1150. The rendering of the display can include rendering video data, analysis data, emotion cluster data, signature data, and so on. The rendering can be displayed on any type of electronic display. The electronic display can include a computer monitor, a laptop display, a tablet display, a smartphone display, a wearable display, a mobile display, a television, a projector and so on. Various steps in the flow 1100 may be changed in order, repeated, omitted, or the like without departing from the disclosed concepts. Various embodiments of the flow 1100 may be included in a computer program product embodied in a non-transitory computer readable medium that includes code executable by one or more processors.

The human face provides a powerful communications medium through its ability to exhibit a myriad of expressions that can be captured and analyzed for a variety of purposes. In some cases, media producers are acutely interested in evaluating the effectiveness of message delivery by video media. Such video media includes advertisements, political messages, educational materials, television programs, movies, government service announcements, etc. Automated facial analysis can be performed on one or more video frames containing a face in order to detect facial action. Based on the facial action detected, a variety of parameters can be determined including affect valence, spontaneous reactions, facial action units, and so on. The parameters that are determined can be used to infer or predict emotional and mental states. For example, determined valence can be used to describe the emotional reaction of a viewer to a video media presentation or another type of presentation. Positive valence provides evidence that a viewer is experiencing a favorable emotional response to the video media presentation, while negative valence provides evidence that a viewer is experiencing an unfavorable emotional response to the video media presentation. Other facial data analysis can include the determination of discrete emotional states of the viewer or viewers.

Facial data can be collected from a plurality of people using any of a variety of cameras. A camera can include a webcam, a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. In some embodiments, the person is permitted to “opt-in” to the facial data collection. For example, the person can agree to the capture of facial data using a personal device such as a mobile device or another electronic device by selecting an opt-in choice. Opting-in can then turn on the person's webcam-enabled device and can begin the capture of the person's facial data via a video feed from the webcam or other camera. The video data that is collected can include one or more persons experiencing an event. The one or more persons can be sharing a personal electronic device or can each be using one or more devices for video capture. The videos that are collected can be collected using a web-based framework. The web-based framework can be used to display the video media presentation or event as well as to collect videos from any number of viewers who are online. That is, the collection of videos can be crowdsourced from those viewers who elected to opt-in to the video data collection.

The videos captured from the various viewers who chose to opt-in can be substantially different in terms of video quality, frame rate, etc. As a result, the facial video data can be scaled, rotated, and otherwise adjusted to improve consistency. Human factors further play into the capture of the facial video data. The facial data that is captured might or might not be relevant to the video media presentation being displayed. For example, the viewer might not be paying attention, might be fidgeting, might be distracted by an object or event near the viewer, or otherwise inattentive to the video media presentation. The behavior exhibited by the viewer can prove challenging to analyze due to viewer actions including eating, speaking to another person or persons, speaking on the phone, etc. The videos collected from the viewers might also include other artifacts that pose challenges during the analysis of the video data. The artifacts can include such items as eyeglasses (because of reflections), eye patches, jewelry, and clothing that occludes or obscures the viewer's face. Similarly, a viewer's hair or hair covering can present artifacts by obscuring the viewer's eyes and/or face.

The captured facial data can be analyzed using the facial action coding system (FACS). The FACS seeks to define groups or taxonomies of facial movements of the human face. The FACS encodes movements of individual muscles of the face, where the muscle movements often include slight, instantaneous changes in facial appearance. The FACS encoding is commonly performed by trained observers, but can also be performed on automated, computer-based systems. Analysis of the FACS encoding can be used to determine emotions of the persons whose facial data is captured in the videos. The FACS is used to encode a wide range of facial expressions that are anatomically possible for the human face. The FACS encodings include action units (AUs) and related temporal segments that are based on the captured facial expression. The AUs are open to higher order interpretation and decision-making. For example, the AUs can be used to recognize emotions experienced by the observed person. Emotion-related facial actions can be identified using the emotional facial action coding system (EMFACS) and the facial action coding system affect interpretation dictionary (FACSAID), for example. For a given emotion, specific action units can be related to the emotion. For example, the emotion of anger can be related to AUs 4, 5, 7, and 23, while happiness can be related to AUs 6 and 12. Other mappings of emotions to AUs have also been previously associated. The coding of the AUs can include an intensity scoring that ranges from A (trace) to E (maximum). The AUs can be used for analyzing images to identify patterns indicative of a particular mental and/or emotional state. The AUs range in number from 0 (neutral face) to 98 (fast up-down look). The AUs include so-called main codes (inner brow raiser, lid tightener, etc.), head movement codes (head turn left, head up, etc.), eye movement codes (eyes turned left, eyes up, etc.), visibility codes (eyes not visible, entire face not visible, etc.), and gross behavior codes (sniff, swallow, etc.). Emotion scoring can be included where intensity is evaluated as well as specific emotions, moods, or mental states.

The coding of faces identified in videos captured of people observing an event can be automated. The automated systems can detect facial AUs or discrete emotional states. The emotional states can include amusement, fear, anger, disgust, surprise, and sadness, for example. The automated systems can be based on a probability estimate from one or more classifiers, where the probabilities can correlate with an intensity of an AU or an expression. The classifiers can be used to identify into which of a set of categories a given observation can be placed. For example, the classifiers can be used to determine a probability that a given AU or expression is present in a given frame of a video. The classifiers can be used as part of a supervised machine learning technique where the machine learning technique can be trained using “known good” data. Once trained, the machine learning technique can proceed to classify new data that is captured.

The supervised machine learning models can be based on support vector machines (SVMs). An SVM can have an associated learning model that is used for data analysis and pattern analysis. For example, an SVM can be used to classify data that can be obtained from collected videos of people experiencing a media presentation. An SVM can be trained using “known good” data that is labeled as belonging to one of two categories (e.g. smile and no-smile). The SVM can build a model that assigns new data into one of the two categories. The SVM can construct one or more hyperplanes that can be used for classification. The hyperplane that has the largest distance from the nearest training point can be determined to have the best separation. The largest separation can improve the classification technique by increasing the probability that a given data point can be properly classified.

In another example, a histogram of oriented gradients (HoG) can be computed. The HoG can include feature descriptors and can be computed for one or more facial regions of interest. The regions of interest of the face can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example. The gradients can be intensity gradients and can be used to describe an appearance and a shape of a local object. The HoG descriptors can be determined by dividing an image into small, connected regions, also called cells. A histogram of gradient directions or edge orientations can be computed for pixels in the cell. Histograms can be contrast-normalized based on intensity across a portion of the image or the entire image, thus reducing any influence from illumination or shadowing changes between and among video frames. The HoG can be computed on the image or on an adjusted version of the image, where the adjustment of the image can include scaling, rotation, etc. For example, the image can be adjusted by flipping the image around a vertical line through the middle of a face in the image. The symmetry plane of the image can be determined from the tracker points and landmarks of the image.

In an embodiment, an automated facial analysis system identifies five facial actions or action combinations in order to detect spontaneous facial expressions for media research purposes. Based on the facial expressions that are detected, a determination can be made with regard to the effectiveness of a given video media presentation, for example. The system can detect the presence of the AUs or the combination of AUs in videos collected from a plurality of people. The facial analysis technique can be trained using a web-based framework to crowdsource videos of people as they watch online video content. The video can be streamed at a fixed frame rate to a server. Human labelers can code for the presence or absence of facial actions including symmetric smile, unilateral smile, asymmetric smile, and so on. The trained system can then be used to automatically code the facial data collected from a plurality of viewers experiencing video presentations (e.g. television programs).

Spontaneous asymmetric smiles can be detected in order to understand viewer experiences. Related literature indicates that as many asymmetric smiles occur on the right hemi face as do on the left hemi face, for spontaneous expressions. Detection can be treated as a binary classification problem, where images that contain a right asymmetric expression are used as positive (target class) samples and all other images as negative (non-target class) samples. Classifiers perform the classification, including classifiers such as support vector machines (SVM) and random forests. Random forests can include ensemble-learning methods that use multiple learning algorithms to obtain better predictive performance. Frame-by-frame detection can be performed to recognize the presence of an asymmetric expression in each frame of a video. Facial points can be detected, including the top of the mouth and the two outer eye corners. The face can be extracted, cropped and warped into a pixel image of specific dimension (e.g. 96×96 pixels). In embodiments, the inter-ocular distance and vertical scale in the pixel image are fixed. Feature extraction can be performed using computer vision software such as OpenCV™. Feature extraction can be based on the use of HoGs. HoGs can include feature descriptors and can be used to count occurrences of gradient orientation in localized portions or regions of the image. Other techniques can be used for counting occurrences of gradient orientation, including edge orientation histograms, scale-invariant feature transformation descriptors, etc. The AU recognition tasks can also be performed using Local Binary Patterns (LBP) and Local Gabor Binary Patterns (LGBP). The HoG descriptor represents the face as a distribution of intensity gradients and edge directions, and is robust in its ability to translate and scale. Differing patterns, including groupings of cells of various sizes and arranged in variously sized cell blocks, can be used. For example, 4×4 cell blocks of 8×8 pixel cells with an overlap of half of the block can be used. Histograms of channels can be used, including nine channels or bins evenly spread over 0-180 degrees. In this example, the HoG descriptor on a 96×96 image is 25 blocks×16 cells×9 bins=3600, the latter quantity representing the dimension. AU occurrences can be rendered. The videos can be grouped into demographic datasets based on nationality and/or other demographic parameters for further detailed analysis.

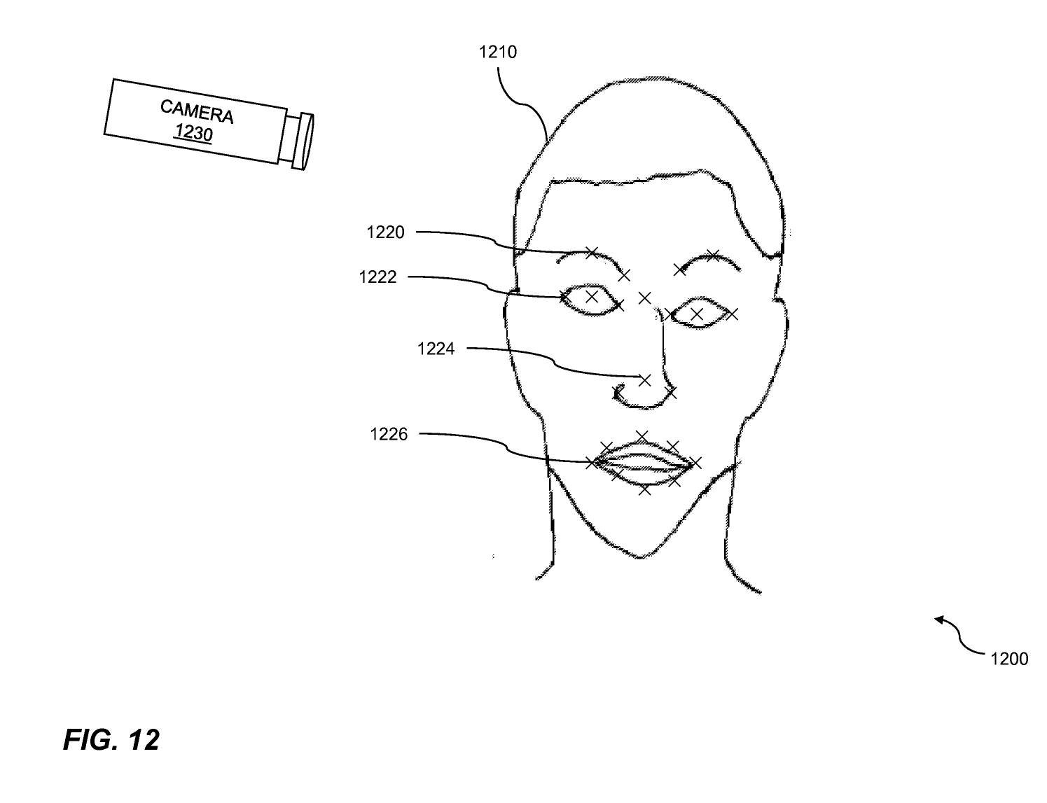

FIG. 12 shows a diagram 1200 illustrating example facial data collection including landmarks. A face 1210 can be observed using a camera 1230 in order to collect facial data that includes facial landmarks. The facial data can be collected from a plurality of people using one or more of a variety of cameras. As discussed above, the camera or cameras can include a webcam, where a webcam can include a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. The quality and usefulness of the facial data that is captured can depend, for example, on the position of the camera 1230 relative to the face 1210, the number of cameras used, the illumination of the face, etc. For example, if the face 1210 is poorly lit or over-exposed (e.g. in an area of bright light), the processing of the facial data to identify facial landmarks might be rendered more difficult. In another example, the camera 1230 being positioned to the side of the person might prevent capture of the full face. Other artifacts can degrade the capture of facial data. For example, the person's hair, prosthetic devices (e.g. glasses, an eye patch, and eye coverings), jewelry, and clothing can partially or completely occlude or obscure the person's face. Data relating to various facial landmarks can include a variety of facial features. The facial features can comprise an eyebrow 1220, an outer eye edge 1222, a nose 1224, a corner of a mouth 1226, and so on. Any number of facial landmarks can be identified from the facial data that is captured. The facial landmarks that are identified can be analyzed to identify facial action units. For example, the action units that can be identified include AU02 outer brow raiser, AU14 dimpler, AU17 chin raiser, and so on. Any number of action units can be identified. The action units can be used alone and/or in combination to infer one or more mental states and emotions. A similar process can be applied to gesture analysis (e.g. hand gestures).

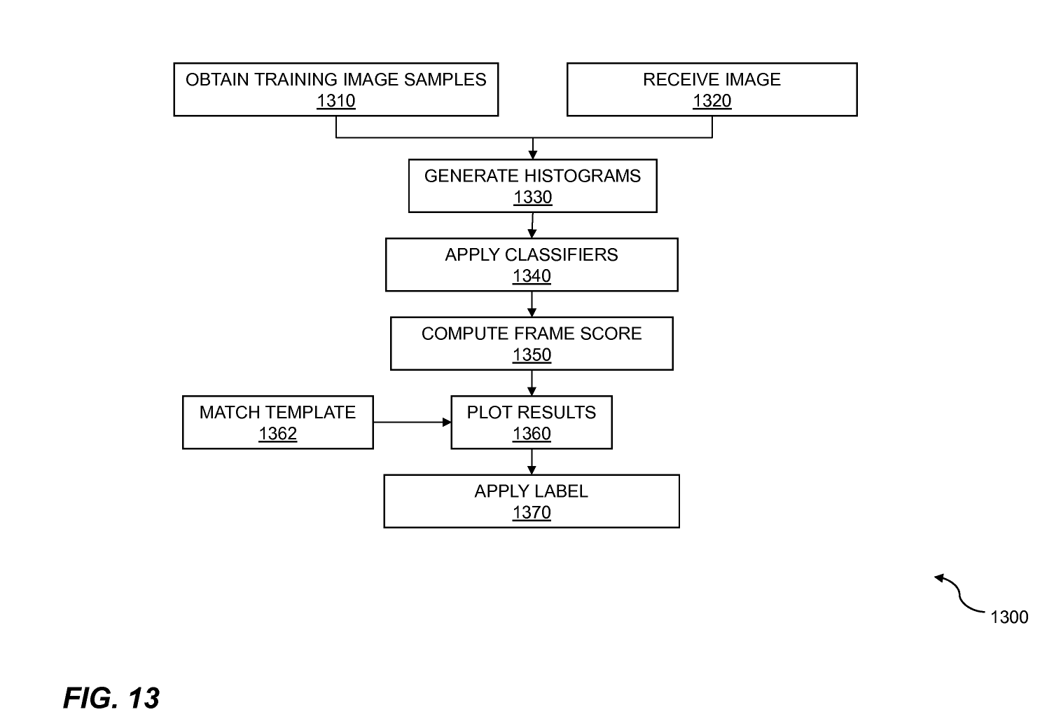

FIG. 13 is a flow for detecting facial expressions. The flow 1300 can be used to automatically detect a wide range of facial expressions. A facial expression can produce strong emotional signals that can indicate valence and discrete emotional states. The discrete emotional states can include contempt, doubt, defiance, happiness, fear, anxiety, and so on. The detection of facial expressions can be based on the location of facial landmarks. The detection of facial expressions can be based on determination of action units (AU) where the action units are determined using FACS coding. The AUs can be used singly or in combination to identify facial expressions. Based on the facial landmarks, one or more AUs can be identified by number and intensity. For example, AU12 can be used to code a lip corner puller and can be used to infer a smirk.

The flow 1300 begins by obtaining training image samples 1310. The image samples can include a plurality of images of one or more people. Human coders who are trained to correctly identify AU codes based on the FACS can code the images. The training or “known good” images can be used as a basis for training a machine learning technique. Once trained, the machine learning technique can be used to identify AUs in other images that can be collected using a camera, such as the camera 1230 from FIG. 4, for example. The flow 1300 continues with receiving an image 1320. The image 1320 can be received from the camera 1230. As discussed above, the camera or cameras can include a webcam, where a webcam can include a video camera, a still camera, a thermal imager, a CCD device, a phone camera, a three-dimensional camera, a depth camera, a light field camera, multiple webcams used to show different views of a person, or any other type of image capture apparatus that can allow captured data to be used in an electronic system. The image 1320 that is received can be manipulated in order to improve the processing of the image. For example, the image can be cropped, scaled, stretched, rotated, flipped, etc. in order to obtain a resulting image that can be analyzed more efficiently. Multiple versions of the same image can be analyzed. For example, the manipulated image and a flipped or mirrored version of the manipulated image can be analyzed alone and/or in combination to improve analysis. The flow 1300 continues with generating histograms 1330 for the training images and the one or more versions of the received image. The histograms can be generated for one or more versions of the manipulated received image. The histograms can be based on a HoG or another histogram. As described above, the HoG can include feature descriptors and can be computed for one or more regions of interest in the training images and the one or more received images. The regions of interest in the images can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example.

The flow 1300 continues with applying classifiers 1340 to the histograms. The classifiers can be used to estimate probabilities where the probabilities can correlate with an intensity of an AU or an expression. The choice of classifiers used is based on the training of a supervised learning technique to identify facial expressions, in some embodiments. The classifiers can be used to identify into which of a set of categories a given observation can be placed. For example, the classifiers can be used to determine a probability that a given AU or expression is present in a given image or frame of a video. In various embodiments, the one or more AUs that are present include AU01 inner brow raiser, AU12 lip corner puller, AU38 nostril dilator, and so on. In practice, the presence or absence of any number of AUs can be determined. The flow 1300 continues with computing a frame score 1350. The score computed for an image, where the image can be a frame from a video, can be used to determine the presence of a facial expression in the image or video frame. The score can be based on one or more versions of the image 1320 or manipulated image. For example, the score can be based on a comparison of the manipulated image to a flipped or mirrored version of the manipulated image. The score can be used to predict a likelihood that one or more facial expressions are present in the image. The likelihood can be based on computing a difference between the outputs of a classifier used on the manipulated image and on the flipped or mirrored image, for example. The classifier that is used can be used to identify symmetrical facial expressions (e.g. smile), asymmetrical facial expressions (e.g. outer brow raiser), and so on.

The flow 1300 continues with plotting results 1360. The results that are plotted can include one or more scores for one or frames computed over a given time t. For example, the plotted results can include classifier probability results from analysis of HoGs for a sequence of images and video frames. The plotted results can be matched with a template 1362. The template can be temporal and can be represented by a centered box function or another function. A best fit with one or more templates can be found by computing a minimum error. Other best-fit techniques can include polynomial curve fitting, geometric curve fitting, and so on. The flow 1300 continues with applying a label 1370. The label can be used to indicate that a particular facial expression has been detected in the one or more images or video frames which constitute the image 1320. For example, the label can be used to indicate that any of a range of facial expressions has been detected, including a smile, an asymmetric smile, a frown, and so on.

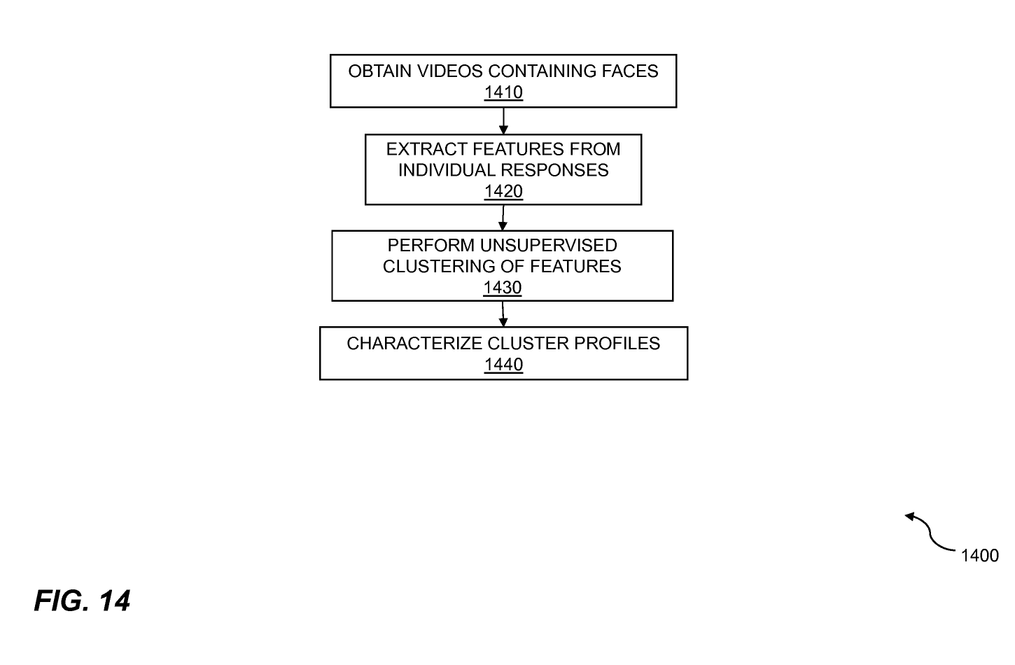

FIG. 14 is a flow 1400 for the large-scale clustering of facial events. As discussed above, collection of facial video data from one or more people can include a web-based framework. The web-based framework can be used to collect facial video data from, for example, large numbers of people located over a wide geographic area. The web-based framework can include an opt-in feature that allows people to agree to facial data collection. The web-based framework can be used to render and display data to one or more people and can collect data from the one or more people. For example, the facial data collection can be based on showing one or more viewers a video media presentation through a website. The web-based framework can be used to display the video media presentation or event and to collect videos from any number of viewers who are online. That is, the collection of videos can be crowdsourced from those viewers who elected to opt-in to the video data collection. The video event can be a commercial, a political ad, an educational segment, and so on. The flow 1400 begins with obtaining videos containing faces 1410. The videos can be obtained using one or more cameras, where the cameras can include a webcam coupled to one or more devices employed by the one or more people using the web-based framework. The flow 1400 continues with extracting features from the individual responses 1420. The individual responses can include videos containing faces observed by the one or more webcams. The features that are extracted can include facial features such as an eyebrow, a nostril, an eye edge, a mouth edge, and so on. The feature extraction can be based on facial coding classifiers, where the facial coding classifiers output a probability that a specified facial action has been detected in a given video frame. The flow 1400 continues with performing unsupervised clustering of features 1430. The unsupervised clustering can be based on an event. The unsupervised clustering can be based on a K-Means, where the K of the K-Means can be computed using a Bayesian Information Criterion (BICk), for example, to determine the smallest value of K that meets system requirements. Any other criterion for K can be used. The K-Means clustering technique can be used to group one or more events into various respective categories.

The flow 1400 continues with characterizing cluster profiles 1440. The profiles can include a variety of facial expressions such as smiles, asymmetric smiles, eyebrow raisers, eyebrow lowerers, etc. The profiles can be related to a given event. For example, a humorous video can be displayed in the web-based framework and the video data of people who have opted-in can be collected. The characterization of the collected and analyzed video can depend in part on the number of smiles that occurred at various points throughout the humorous video. Similarly, the characterization can be performed on collected and analyzed videos of people viewing a news presentation. The characterized cluster profiles can be further analyzed based on demographic data. For example, the number of smiles resulting from people viewing a humorous video can be compared to various demographic groups, where the groups can be formed based on geographic location, age, ethnicity, gender, and so on.

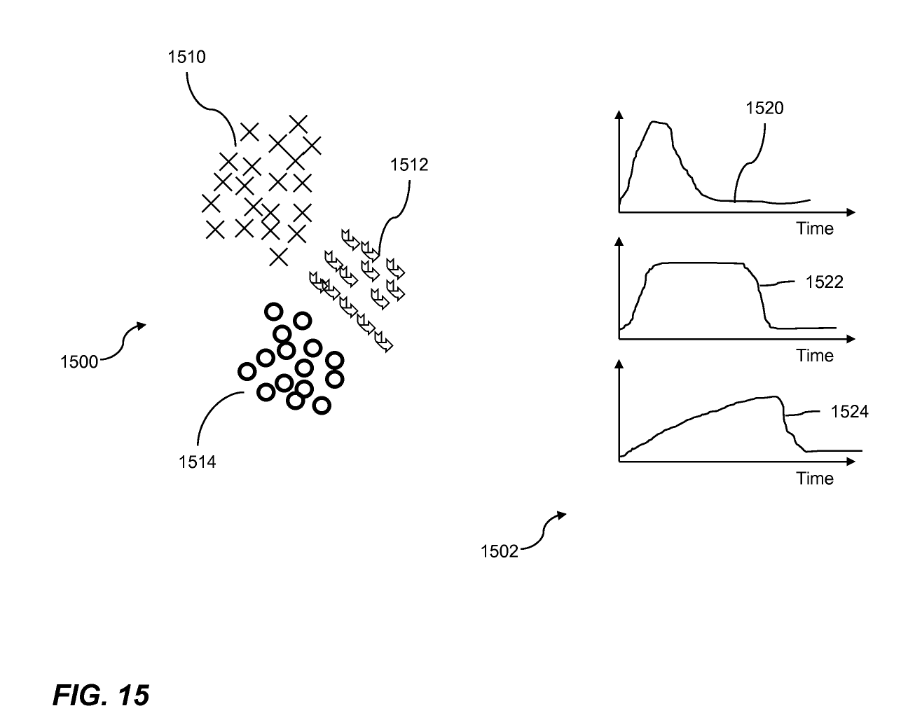

FIG. 15 shows example unsupervised clustering of features and characterization of cluster profiles. Features including samples of facial data can be clustered using unsupervised clustering. Various clusters can be formed, which include similar groupings of facial data observations. The example 1500 shows three clusters 1510, 1512, and 1514. The clusters can be based on video collected from people who have opted-in to video collection. When the data collected is captured using a web-based framework, then the data collection can be performed on a grand scale, including hundreds, thousands, or even more participants who can be located locally and/or across a wide geographic area. Unsupervised clustering is a technique that can be used to process the large amounts of captured facial data and to identify groupings of similar observations. The unsupervised clustering can also be used to characterize the groups of similar observations. The characterizations can include identifying behaviors of the participants. The characterizations can be based on identifying facial expressions and facial action units of the participants. Some behaviors and facial expressions can include faster or slower onsets, faster or slower offsets, longer or shorter durations, etc. The onsets, offsets, and durations can all correlate to time. The data clustering that results from the unsupervised clustering can support data labeling. The labeling can include FACS coding. The clusters can be partially or totally based on a facial expression resulting from participants viewing a video presentation, where the video presentation can be an advertisement, a political message, educational material, a public service announcement, and so on. The clusters can be correlated with demographic information, where the demographic information can include educational level, geographic location, age, gender, income level, and so on.

Cluster profiles 1502 can be generated based on the clusters that can be formed from unsupervised clustering, with time shown on the x-axis and intensity or frequency shown on the y-axis. The cluster profiles can be based on captured facial data including facial expressions, for example. The cluster profile 1520 can be based on the cluster 1510, the cluster profile 1522 can be based on the cluster 1512, and the cluster profile 1524 can be based on the cluster 1514. The cluster profiles 1520, 1522, and 1524 can be based on smiles, smirks, frowns, or any other facial expression. Emotional states of the people who have opted-in to video collection can be inferred by analyzing the clustered facial expression data. The cluster profiles can be plotted with respect to time and can show a rate of onset, a duration, and an offset (rate of decay). Other time-related factors can be included in the cluster profiles. The cluster profiles can be correlated with demographic information as described above.

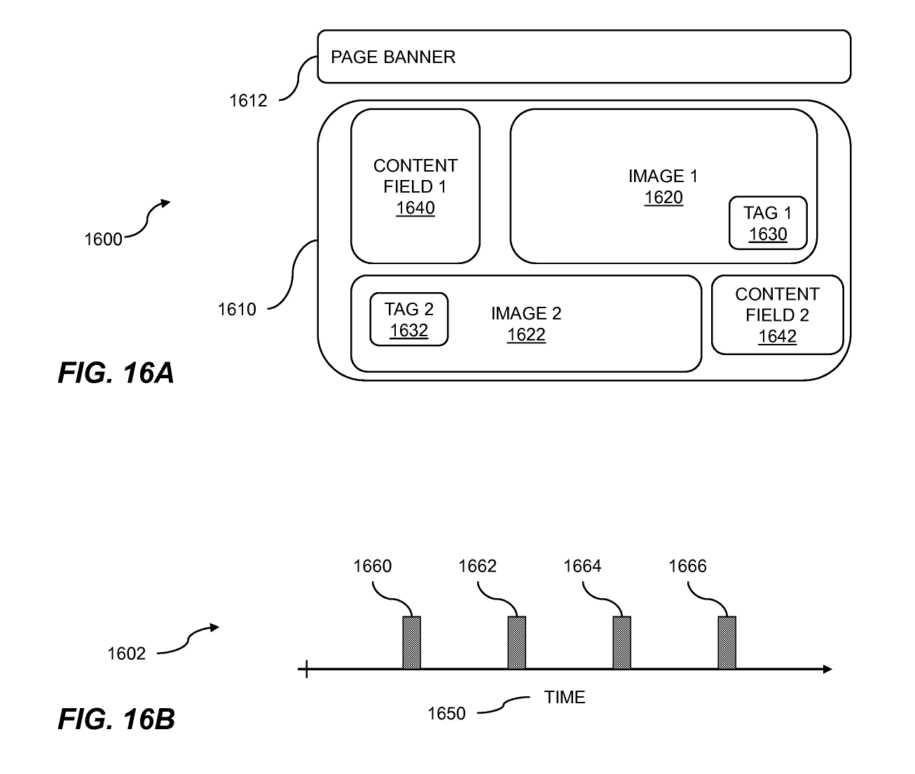

FIG. 16A shows example tags embedded in a webpage. A webpage 1600 can include a page body 1610, a page banner 1612, and so on. The page body can include one or more objects, where the objects can include text, images, videos, audio, and so on. The example page body 1610 shown includes a first image, image 1 1620; a second image, image 2 1622; a first content field, content field 1 1640; and a second content field, content field 2 1642. In practice, the page body 1610 can contain any number of images and content fields, and can include one or more videos, one or more audio presentations, and so on. The page body can include embedded tags, such as tag 1 1630 and tag 2 1632. In the example shown, tag 1 1630 is embedded in image 1 1620, and tag 2 1632 is embedded in image 2 1622. In embodiments, any number of tags can be imbedded. Tags can also be imbedded in content fields, in videos, in audio presentations, etc. When a user mouses over a tag or clicks on an object associated with a tag, the tag can be invoked. For example, when the user mouses over tag 1 1630, tag 1 1630 can then be invoked. Invoking tag 1 1630 can include enabling a camera coupled to a user's device and capturing one or more images of the user as the user views a media presentation (or digital experience). In a similar manner, when the user mouses over tag 2 1632, tag 2 1632 can be invoked. Invoking tag 2 1632 can also include enabling the camera and capturing images of the user. In other embodiments, other actions can be taken based on invocation of the one or more tags. For example, invoking an embedded tag can initiate an analysis technique, post to social media, award the user a coupon or another prize, initiate mental state analysis, perform emotion analysis, and so on.

FIG. 16B shows example tag invoking to collect images. As stated above, a media presentation can be a video, a webpage, and so on. A video 1602 can include one or more embedded tags, such as a tag 1660, another tag 1662, a third tag 1664, a fourth tag 1666, and so on. In practice, any number of tags can be included in the media presentation. The one or more tags can be invoked during the media presentation. The collection of the invoked tags can occur over time as represented by a timeline 1650. When a tag is encountered in the media presentation, the tag can be invoked. For example, when the tag 1660 is encountered, invoking the tag can enable a camera coupled to a user device and can capture one or more images of the user viewing the media presentation. Invoking a tag can depend on opt-in by the user. For example, if a user has agreed to participate in a study by indicating an opt-in, then the camera coupled to the user's device can be enabled and one or more images of the user can be captured. If the user has not agreed to participate in the study and has not indicated an opt-in, then invoking the tag 1660 does not enable the camera nor capture images of the user during the media presentation. The user can indicate an opt-in for certain types of participation, where opting-in can be dependent on specific content in the media presentation. For example, the user could opt-in to participation in a study of political campaign messages and not opt-in for a particular advertisement study. In this case, tags that are related to political campaign messages and that enable the camera and image capture when invoked would be embedded in the media presentation. However, tags imbedded in the media presentation that are related to advertisements would not enable the camera when invoked. Various other situations of tag invocation are possible.

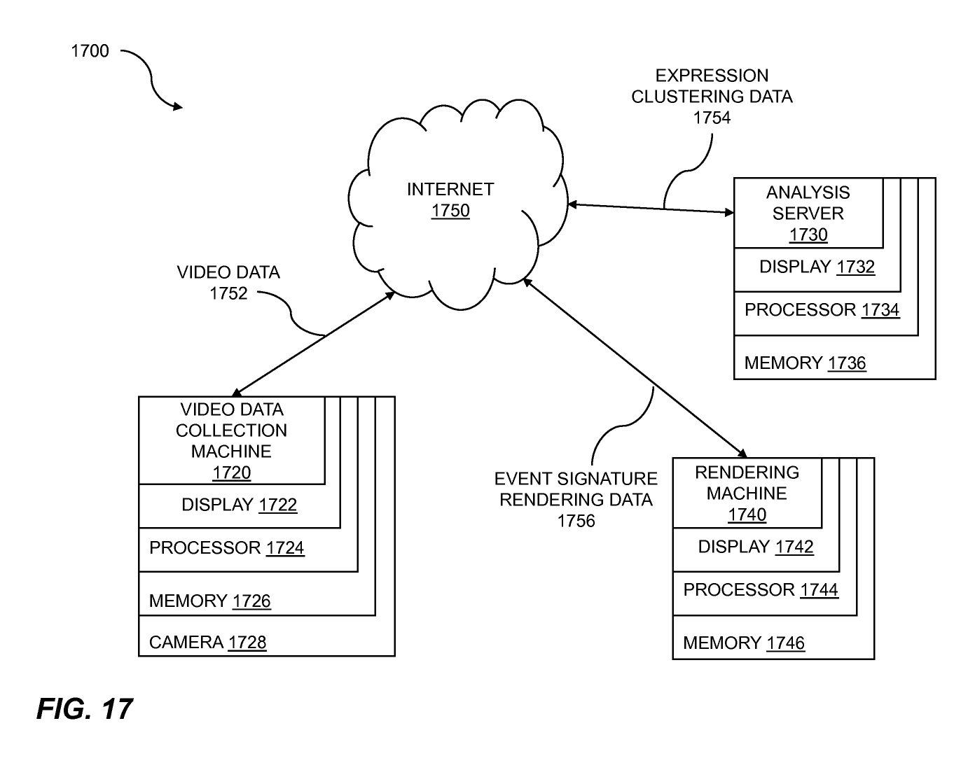

FIG. 17 is a system 1700 for mental state event definition generation. An example system 1700 is shown for mental state event definition collection, analysis, and rendering. The system 1700 can include a memory which stores instructions and one or more processors attached to the memory wherein the one or more processors, when executing the instructions which are stored, are configured to: obtain a plurality of videos of people; analyze the plurality of videos using classifiers; perform expression clustering based on the analyzing; and determine a temporal signature for an event based on the expression clustering.

The system 1700 can provide a computer-implemented method for analysis comprising: receiving information on a plurality of videos of people; analyzing the plurality of videos using classifiers; performing expression clustering based on the analyzing; and determining a temporal signature for an event based on the expression clustering.

The system 1700 can provide a computer-implemented method for analysis comprising: receiving classifiers for facial expressions, obtaining a plurality of videos of people, analyzing the plurality of videos using classifiers, performing expression clustering based on the analyzing, and determining a temporal signature for an event based on the expression clustering.

The system 1700 can include one or more video data collection machines 1720 linked to an analysis server 1730 and a rendering machine 1740 via the Internet 1750 or another computer network. The network can be wired or wireless. Video data 1752 can be transferred to the analysis server 1730 through the Internet 1750, for example. The example video collection machine 1720 shown comprises one or more processors 1724 coupled to a memory 1726 which can store and retrieve instructions, a display 1722, and a camera 1728. The camera 1728 can include a webcam, a video camera, a still camera, a thermal imager, a CCD device, a phone camera, a three-dimensional camera, a depth camera, a light field camera, multiple webcams used to show different views of a person, or any other type of image capture technique that can allow captured data to be used in an electronic system. The memory 1726 can be used for storing instructions, video data on a plurality of people, one or more classifiers, and so on. The display 1722 can be any electronic display, including but not limited to, a computer display, a laptop screen, a net-book screen, a tablet computer screen, a smartphone display, a mobile device display, a remote with a display, a television, a projector, or the like.

The analysis server 1730 can include one or more processors 1734 coupled to a memory 1736 which can store and retrieve instructions, and can also include a display 1732. The analysis server 1730 can receive the video data 1752 and analyze the video data using classifiers. The classifiers can be stored in the analysis server, loaded into the analysis server, provided by a user of the analysis server, and so on. The analysis server 1730 can use video data received from the video data collection machine 1720 to produce expression-clustering data 1754. In some embodiments, the analysis server 1730 receives video data from a plurality of video data collection machines, aggregates the video data, processes the video data or the aggregated video data, and so on.

The rendering machine 1740 can include one or more processors 1744 coupled to a memory 1746 which can store and retrieve instructions and data, and can also include a display 1742. The rendering of event signature rendering data 1756 can occur on the rendering machine 1740 or on a different platform than the rendering machine 1740. In embodiments, the rendering of the event signature rendering data can occur on the video data collection machine 1720 or on the analysis server 1730. As shown in the system 1700, the rendering machine 1740 can receive event signature rendering data 1756 via the Internet 1750 or another network from the video data collection machine 1720, from the analysis server 1730, or from both. The rendering can include a visual display or any other appropriate display format. The system 1700 can include a computer program product embodied in a non-transitory computer readable medium for analysis comprising: code for obtaining a plurality of videos of people, code for analyzing the plurality of videos using classifiers, code for performing expression clustering based on the analyzing, and code for determining a temporal signature for an event based on the expression clustering.

Each of the above methods may be executed on one or more processors on one or more computer systems. Embodiments may include various forms of distributed computing, client/server computing, and cloud based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”— may be implemented by computer program instructions, by special-purpose hardware-based computer systems, by combinations of special purpose hardware and computer instructions, by combinations of general purpose hardware and computer instructions, and so on.

A programmable apparatus which executes any of the above mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the forgoing examples should not limit the spirit and scope of the present invention; rather it should be understood in the broadest sense allowable by law.

What is claimed is:

1. A computer-implemented method for analysis comprising:
obtaining a plurality of videos of people;
analyzing the plurality of videos using classifiers;
performing expression clustering based on the analyzing; and
determining a temporal signature for an event based on the expression clustering.

2. The method of claim 1 wherein the temporal signature includes a length.

3. The method of claim 2 wherein the length is computed based on detection of adjacent local minima of a facial expression probability curve.

4. The method of claim 1 wherein the temporal signature includes a peak intensity.

5. The method of claim 1 wherein the temporal signature includes a shape for an intensity transition from low intensity to a peak intensity.

6. The method of claim 1 wherein the temporal signature includes a shape for an intensity transition from a peak intensity to low intensity.

7. The method of claim 1 wherein the plurality of videos are of people who are viewing substantially identical situations that include viewing media.

8. The method of claim 7 wherein the media is oriented toward an emotion.

9. The method of claim 8 wherein the emotion includes one or more of humor, sadness, poignancy, and mirth.

10. (canceled)

11. The method of claim 1 wherein the temporal signature includes a peak intensity and a rise rate to the peak intensity.

12. The method of claim 11 further comprising filtering events having the peak intensity less than a predetermined threshold.

13. (canceled)

14. The method of claim 1 wherein the temporal signature includes a rise rate, a peak intensity, and a decay rate.

15. The method of claim 14 wherein the analyzing further comprises classifying a facial expression as belonging to a category of posed or spontaneous.

16. The method of claim 1 wherein a classifier, from the classifiers, is used on a mobile device where the plurality of videos are obtained with the mobile device.

17. The method of claim 1 wherein the expression clustering is for smiles, smirks, brow furrows, squints, lowered eyebrows, raised eyebrows, or attention.

18. The method of claim 1 wherein the expression clustering is for inner brow raiser, outer brow raiser, brow lowerer, upper lid raiser, cheek raiser, lid tightener, lips toward each other, nose wrinkle, upper lid raiser, nasolabial deepener, lip corner puller, sharp lip puller, dimpler, lip corner depressor, lower lip depressor, chin raiser, lip pucker, tongue show, lip stretcher, neck tightener, lip funneler, lip tightener, lips part, jaw drop, mouth stretch, lip suck, jaw thrust, jaw sideways, jaw clencher, lip bite, cheek blow, cheek puff, cheek suck, tongue bulge, lip wipe, nostril dilator, nostril compressor, glabella lowerer, inner eyebrow lowerer, eyes closed, eyebrow gatherer, blink, wink, head turn left, head turn right, head up, head down, head tilt left, head tilt right, head forward, head thrust forward, head back, head shake up and down, head shake side to side, head upward and to a side, eyes turn left, eyes left, eyes turn right, eyes right, eyes up, eyes down, walleye, cross-eye, upward rolling of eyes, clockwise upward rolling of eyes, counter-clockwise upward rolling of eyes, eyes positioned to look at other person, head and/or eyes look at other person, sniff, speech, swallow, chewing, shoulder shrug, head shake back and forth, head nod up and down, flash, partial flash, shiver/tremble, or fast up-down look.

19. The method of claim 1 wherein the expression clustering is for a combination of facial expressions.

20. The method of claim 1 further comprising using the temporal signature to infer a mental state where the mental state includes one or more of sadness, stress, anger, happiness, disgust, frustration, confusion, disappointment, hesitation, cognitive overload, focusing, engagement, attention, boredom, exploration, confidence, trust, delight, skepticism, doubt, satisfaction, excitement, laughter, calmness, and curiosity.

21. The method of claim 1 wherein the analyzing includes:
identifying a human face within a frame of a video selected from the plurality of videos;
defining a region of interest (ROI) in the frame that includes the identified human face;
extracting one or more histogram-of-oriented-gradients (HoG) features from the ROI; and
computing a set of facial metrics based on the one or more HoG features.

22. The method of claim 21 further comprising smoothing each metric from the set of facial metrics.

23. (canceled)

24. The method of claim 1 wherein the performing expression clustering comprises performing K-means clustering.

25. (canceled)

26. The method of claim 1 further comprising associating demographic information with each event.

27. The method of claim 26 wherein the demographic information includes country of residence.

28. The method of claim 27 further comprising generating an international event signature profile.

29. The method of claim 1 wherein the analyzing includes:
identifying multiple human faces within a frame of a video selected from the plurality of videos;
defining a region of interest (ROI) in the frame for each identified human face;
extracting one or more histogram-of-oriented-gradients (HoG) features from each ROI; and
computing a set of facial metrics based on the one or more HoG features for each of the multiple human faces.

30. A computer program product embodied in a non-transitory computer readable medium for analysis, the computer program product comprising:
code for obtaining a plurality of videos of people;
code for analyzing the plurality of videos using classifiers;
code for performing expression clustering based on the analyzing; and
code for determining a temporal signature for an event based on the expression clustering.

31. A computer system for analysis comprising:
a memory which stores instructions;
one or more processors attached to the memory wherein the one or more processors, when executing the instructions which are stored, are configured to:
obtain a plurality of videos of people;
analyze the plurality of videos using classifiers;
perform expression clustering based on the analyzing; and
determine a temporal signature for an event based on the expression clustering.

32-33. (canceled)
